(19) United States
(12) Patent Application Publication
(10) Pub. No.: US 2004/0048312 A1
(43) Pub. Date: Mar. 11, 2004

(54) Antibodies that bind to integrin alpha-v-beta-6 and methods of use thereof

(76) Inventors: Ronghao Li, Millbrae, CA (US); Jennie P. Mather, Millbrae, CA (US); Zhuangyu Pan, Millbrae, CA (US)

Correspondence Address: Jie Zhou, Morrison & Foerster LLP, 755 Page Mill Road, Palo Alto, CA, 94304-1018, US

(21) Appl. No.: 10/414,176

(22) Filed: Apr. 14, 2003

Related U.S. Application Data
(60) Provisional application No. 60/372,277, filed on Apr. 12, 2002.
(60) Provisional application No. 60/373,274, filed on Apr. 16, 2002.

(51) Int. Cl. G01N 33/53; C07H 21/04; C07K 16/28; C07K 16/46; C12N 5/06

(52) U.S. Cl. 435/7.1; 435/69.1; 435/320.1; 435/334; 530/388.22; 536/23.53

(57) ABSTRACT

The invention provides monoclonal antibody mBLA3 that binds to integrin αvβ6 and other equivalent antibodies. The invention also provides the identification and characterization of cancer-associated antigen, integrin αvβ6, with various human cancers and methods of diagnosing and treating these human cancers.

### CROSS-REFERENCE TO RELATED APPLICATIONS

This application claims the benefit of U.S. provisional application serial No. 60/372,277, filed Apr. 12, 2002, and No. 60/373,274, filed Apr. 16, 2002, which are incorporated in their entirety by reference.

### TECHNICAL FIELD

This invention is in the fields of cancer biology and immunotherapy. More specifically, it concerns the discovery of antibodies such as monoclonal antibody mBLA3 that bind to integrin alpha-v-beta-6, which is present in a variety of human cancers, methods of diagnosing and/or treating such cancers.

### BACKGROUND OF THE INVENTION

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6^{th} Edition (2001) Chapt. 20 pp. 495-508. These antibodies can have inherent therapeutic biological activity both by direct inhibition of tumor cell growth or survival and by their ability to recruit the natural cell killing activity of the body's immune system. These agents can be administered alone or in conjunction with radiation or chemotherapeutic agents. Rituxan® and Herceptin®, approved for treatment of lymphoma and breast cancer, respectively, are two examples of such therapeutics. Alternatively, antibodies can be used to make antibody conjugates where the antibody is linked to a toxic agent and directs that agent to the tumor by specifically binding to the tumor. Mylotarg® is an example of an approved antibody conjugate used for the treatment of leukemia. Monoclonal antibodies that bind to cancer cells and have potential uses for diagnosis and therapy have been disclosed in publications. See, for example, the following patent applications which disclose, inter alia, some molecular weights of target proteins: U.S. Pat. No. 6,054,561 (200 kD c-erbB-2 (Her2), and other unknown antigens 40-200 KD in size) and U.S. Pat. No. 5,656,444 (50 kD and 55 kD, oncofetal protein). Example of antibodies in clinical trials and/or approved for treatment of solid tumors include: Herceptin (antigen: 180 kD, HER2/neu), Panorex (antigen: 40-50 kD, Ep-CAM), HMFG1 (antigen >200 kD, HMW Mucin), C225 (antigens: 150 kD and 170 kD, EGF receptor), Campath (antigen: 21-28 kD, CD52), and Rituxan (antigen: 35 kD, CD20).

Another type of immunotherapy is active immunotherapy, or vaccination, wherein the antigen present on a specific cancer(s) or a DNA construct that directs the expression of the antigen, which then evokes the immune response in the individual, i.e., to induce the individual to actively produce antibodies against their own cancer. Active immunization has not been used as often as passive immunotherapy or immunotoxins.

Integrin alpha-v-beta-6 (αvβ6) has been reported to be associated with cancer. See, for example, J. M. Breuss, et al., J. Cell Science 108: 2241-2251 (1995); K. Arihiro, et al., Breast Cancer 7: 19-26 (2000); M. Agrez, et al., Int. J. Cancer 81: 90-97 (1999); M. Busk, et al., J. Biol. Chem. 267: 5790-5796 (1992); J. Jones, et al., J. Oral. Pathol. Med. 26: 63-8 (1997); M. Lehmann, et al., Cancer Res. 54: 2101-7 (1994); M. Agrez, et al., J. Cell. Biol. 127: 547-56 (1994); and R. B. Dixit, et al., J. Biol. Chem. 271(42): 25976-25980 (1996); N. Ahmed, et al., J. Histochem. Cytochem. 50(10):1371-80 (2002). Some monoclonal antibodies to integrin αvβ6 have been made. See, for example, U.S. Pat. No. 6,316,601; M. Lehmann, et al., Cancer Res. 54: 2102-7 (1994); A. Weinacker, et al., J. Biol. Chem. 269: 6940-6948 (1994); J. M. Breuss, et al., J. Cell Science 108: 2241-2251 (1995); K Arihiro, et al., Breast Cancer 7: 19-26 (2000); and J. Jones, et al., J. Oral. Pathol. Med. 26: 63-8 (1997). Other monoclonal antibodies to αv-integrin have been disclosed. See, for example, Mitjans et al. (U.S. Pat. No. 5,985,278). Other monoclonal antibodies have been disclosed for β6 integrins. See, for example, Huang, et al., (U.S. Pat. No. 6,316,601).

Integrins are a super-family of cell surface adhesion molecules that control the attachment of cells to extracellular matrices and other cells. Integrins have important roles in cell migration, growth and differentiation. Integrins are heterodimers consisting of two type I transmembrane glycoproteins, the alpha (cc) and the beta (β), subunits. Currently, there are eight reported β subunits and sixteen cc subunits, pairing into 22 known integrin pairs. Each one of these heterodimeric adhesion molecules have a different ligand binding profile and intra/inter-cellular signaling functions. Specifically, the αv subunit is capable of associating with the β1, β3, β5, and β6 subunits.

Although monoclonal antibodies to integrin αvβ6 have been disclosed, very few antibodies, if any, have been shown to have cancer growth inhibitory properties. What is needed is antibodies to αvβ6, preferably targeting the more restricted β6 subunits, that have cancer growth inhibitory properties.

### SUMMARY OF THE INVENTION

The invention disclosed herein concerns antibodies that bind to β6 integrin subunit, which is expressed on a variety of human cancers. Accordingly, in one aspect, the invention is an antibody or a polypeptide (which may or may not be an antibody) that binds preferentially to β6 integrin subunit. In one embodiment, the antibody or the polypeptide remains immunologically active after denaturation of the target antigen (β6 integrin subunit). In another embodiment, the antibody or the polypeptide is only immunologically active to a non-reduced target antigen (β6 integrin subunit).

In another aspect, the invention is a monoclonal antibody mBLA3 that is produced by a host cell line deposited at American Type Culture Collection with a Patent Deposit Number PTA-3775. The antibody mBLA3 binds to native and denatured β6 integrin subunit only in non-reduced form.

In another aspect, the invention is an antibody or a polypeptide (which may or may not be an antibody) that competitively inhibits preferential binding of mBLA3 to β6 integrin subunit. In some embodiments, the invention is an antibody or a polypeptide (which may or may not be an antibody) that binds preferentially to the same epitope on β6 integrin subunit as mBLA3 binds preferentially. The epitope on β6 integrin subunit that mBLA3 binds is disulfide bond dependent.

In another aspect, the invention is an antibody comprising a fragment or a region of the antibody mBLA3. In one embodiment, the fragment is a light chain of the antibody mBLA3. In another embodiment, the fragment is a heavy chain of the antibody mBLA3. In yet another embodiment, the fragment contains one or more variable regions from a light chain and/or a heavy chain of the antibody mBLA3. In yet another embodiment, the fragment contains one or more complementarity determining regions (CDRS) from a light chain and/or a heavy chain of the antibody mBLA3.

In another aspect, the invention provides polypeptides (which may or may not be antibodies) comprising any of the following: a) one or more CDRs from the light or heavy chain; b) three CDRs from the light chain; c) three CDRs from the heavy chain; d) three CDRs from the light chain and three CDRs from the heavy chain; e) the light chain variable region; f) the heavy chain variable region of the antibody mBLA3.

In another aspect, the invention is a humanized antibody derived from mBLA3. In some embodiments, the humanized antibody comprises one or more CDRs of the antibody mBLA3. In another aspect, the invention provides a humanized antibody that competitively inhibits preferential binding of mBLA3 to β6 integrin subunit. In some embodiments, the humanized antibody binds to the same epitope(s) as antibody mBLA3. Generally, a humanized antibody of the invention comprises one or more (one, two, three, four, five, six) CDRs which are the same and/or derived from the CDR(s) of antibody mBLA3. In other aspect, the invention provides a human antibody that competitively inhibits preferential binding of mBLA3 to β6 integrin subunit. In some embodiments, the human antibody binds to the same epitope(s) on β6 integrin subunit as antibody mBLA3.

In anther aspect, the invention is a chimeric antibody comprising variable regions derived from variable regions of a heavy chain and a light chain of antibody mBLA3 and constant regions derived from constant regions of a heavy chain and a light chain of a human antibody.

In some embodiments, the antibody or the polypeptide described herein are linked to a therapeutic agent (such as a radioactive moiety). In some embodiments, the antibody or the polypeptide described herein are linked to a prodrug-activating enzyme which converts a prodrug to an active anti-cancer drug.

In yet another aspect, the invention is a host cell (ATCC No. PTA-3775) which produces monoclonal antibody mBLA3.

In another aspect, the invention is an isolated polynucleotide that encodes for antibody mBLA3 that is produced by a host cell with a deposit number of ATCC No. PTA-3775 or progeny thereof. In another aspect, the invention provides polynucleotides encoding any of the antibodies (including antibody fragments) as well as any other polypeptides described herein.

In yet another aspect, the invention is an isolated complex comprising β36 integrin subunit bound by an antibody specific for an epitope of β6 integrin subunit. In one embodiment, the antibody is mBLA3. In yet another aspect, the invention is an isolated complex comprising integrin αvβ6 bound by an antibody specific for an epitope of β6 integrin subunit. In one embodiment, the antibody is mBLA3.

In yet another aspect, the invention is a complex comprising β6 integrin subunit bound by any of the antibody or polypeptides described herein. In some embodiments, β6 integrin subunit is associated with αv integrin subunit. In some embodiments, β6 integrin subunit is present on breast, colon, lung, pancreatic, prostate, esophageal, thyroid, renal, or ovarian cancer cells. In some embodiments, the cancer cells are metastatic. In some embodiments, the antibody is mBLA3 or an antibody that competitively inhibits preferential binding of mBLA3 to β6 integrin subunit. In some embodiments, antibody mBLA3 or the antibody that competitively inhibits preferential binding of mBLA3 to β6 integrin subunit are linked to a therapeutic agent (such as a radioactive moiety). In some embodiments, antibody mBLA3 or the antibody that competitively inhibits preferential binding of mBLA3 to β6 integrin subunit are linked to a prodrug-activating enzyme which converts a prodrug to an active anti-cancer drug. In some embodiments, the antibody binds preferentially to the same epitope that mBLA3 binds preferentially. In some embodiments, the complexes described above are isolated.

In another aspect, the invention is a complex of a cancer cell expressing β6 integrin subunit bound by any of the antibody or polypeptide described herein. In some embodiments, β6 integrin subunit is associated with αv integrin subunit. In some embodiments, β6 integrin subunit is present on breast, colon, lung, pancreatic, prostate, esophageal, thyroid, renal, or ovarian cancer cells. In some embodiments, the cancer cells are metastatic. In some embodiments, the antibody is mBLA3 or an antibody that competitively inhibits preferential binding of mBLA3 to β6 integrin subunit. In some embodiments, antibody mBLA3 or the antibody that competitively inhibits preferential binding of mBLA3 to β6 integrin subunit are linked to a therapeutic agent (such as a radioactive moiety). In some embodiments, antibody mBLA3 or the antibody that competitively inhibits preferential binding of mBLA3 to β6 integrin subunit are linked to a prodrug-activating enzyme which converts a prodrug to an active anti-cancer drug. In some embodiments, the antibody binds preferentially to the same epitope that antibody mBLA3 binds preferentially. In some embodiments, the complexes described above are isolated.

In another aspect, the invention is a complex of an epitope on β6 integrin subunit that mBLA3 preferentially binds bound by any of the antibody or polypeptides described herein. In some embodiments, β6 integrin subunit is associated with αv integrin subunit. In some embodiments, β6 integrin subunit is present on breast, colon, lung, pancreatic, prostate, esophageal, thyroid, renal, or ovarian cancer cells. In some embodiments, the cancer cells are metastatic. In some embodiments, the antibody is mBLA3 or an antibody that competitively inhibits preferential binding of mBLA3 to β6 integrin subunit. In some embodiments, antibody mBLA3 or the antibody that competitively inhibits preferential binding of mBLA3 to β6 integrin subunit are linked to a therapeutic agent (such as a radioactive moiety). In some embodiments, antibody mBLA3 or the antibody that competitively inhibits preferential binding of mBLA3 to β6 integrin subunit are linked to a prodrug-activating enzyme which converts a prodrug to an active anti-cancer drug. In some embodiments, the antibody binds preferentially to the same epitope that antibody mBLA3 binds preferentially. In some embodiments, the complexes described above are isolated.

In another aspect, the invention is a pharmaceutical composition comprising any of the polypeptides (including any of the antibodies such as antibody mBLA3) or polynucleotides described herein, such as pharmaceutical compositions comprising the antibody mBLA3, the antibody mBLA3 linked to a therapeutic agent, an antibody comprising a fragment of the antibody mBLA3, a humanized antibody of the antibody mBLA3, a chimeric antibody comprising variable regions derived from variable regions of the antibody mBLA3 and constant regions derived from constant regions of a human antibody, or a human antibody with one or more properties of the antibody mBLA3, or any of the antibody described herein linked to a therapeutic agent (such as a radioactive moiety) or a prodrug-activating enzyme which converts a prodrug to an active anti-cancer drug, and a pharmaceutically acceptable excipient.

In yet another aspect, the invention is a method of generating a monoclonal antibody having greater affinity to cancerous cells than non-cancerous cells comprising the steps of: (a) immunizing a host mammal with intact human fetal bladder cells (HFB); (b) obtaining lymphocytes from the mammal; (c) fusing lymphocytes (b) with a myeloma cell line to produce a hybridoma; (d) culturing the hybridoma of (c) to produce monoclonal antibodies; and (e) screening the antibodies to select only those antibodies which bind to cancerous cells or cell lines but do not bind to non-cancerous cells or cell lines, or bind to normal cells at a lower level or in a different fashion.

In another aspect, the invention is a method of producing antibody mBLA3 comprising culturing a host cell (ATCC No. PTA-3775) or progeny thereof under conditions that allow production of antibody mBLA3, and purifying the antibody mBLA3. In other embodiments, an antibody of the invention is produced by culturing a hybridoma that produces the antibody under conditions that allow production of the antibody. The antibody may then be purified or isolated.

In another aspect, the invention provides methods of producing any of the antibodies (or polypeptides) described herein by expressing one or more polynucleotides encoding the antibody (which may be separately expressed as a single light or heavy chain, or both a light and a heavy chain are expressed from one vector) in a suitable cell, generally followed by recovering and/or isolating the antibody or polypeptides of interest.

In another aspect, the invention is a method of diagnosing (e.g., detecting or identifying presence or absence) cancer in an individual by detecting β6 integrin subunit from cells from the individual using the antibody mBLA3 or any β6 integrin subunit binding moiety (polypeptides, including, but not limited to, various antibodies and antibody derivatives) described herein. In some embodiments, the cancer is breast, colon, lung, pancreatic, prostate, esophageal, thyroid, renal, and ovarian cancer. In some embodiments, the method is detecting the level (or in some embodiments, presence or absence) of β6 integrin subunit from cells. The presence of β6 integrin subunit is detected by detecting a complex between β6 integrin subunit and a β6 integrin subunit binding moiety. The term “detection” as used herein include qualitative and/or quantitative detection (measuring levels) with or without reference to a control.

In yet another aspect, the invention is a method of diagnosing (e.g., detecting or identifying presence or absence) prostate cancer in an individual by detecting antigen β6 integrin subunit from cells from the individual. For these embodiments, any antibody (or moiety which binds to β6 integrin subunit) may be used. In one embodiment, β6 integrin subunit from cells are detected by the antibody mBLA3 or any β6 integrin subunit binding moiety (polypeptides, including, but not limited to, various antibodies and antibody derivatives) described herein. In some embodiments, the prostate cancer is metastatic.

In yet another aspect, the invention is a method of diagnosing metastatic cancer in an individual by detecting antigen β6 integrin subunit from cells from the individual. In some embodiments, the metastatic cancer is breast, prostate, renal, thyroid, esophageal, or ovarian metastatic cancer. In some embodiments, the antigen β6 integrin subunit is detected by detecting a complex of the antigen β6 integrin subunit bound by antibody mBLA3 or any β6 integrin subunit binding moiety (polypeptides, including, but not limited to, various antibodies and antibody derivatives) described herein.

In another aspect, the invention is a method of treating cancer by administering an effective amount of a composition comprising an antibody that binds to β6 integrin subunit sufficient to reduce growth of cancerous cells. In some embodiments, the antibody is the antibody mBLA3, or any of the antibodies (including polypeptides) or polynucleotides embodiments described herein, including but not limited to the antibody mBLA3 associated with a therapeutic agent, an antibody comprising a fragment or a region of the antibody mBLA3, a humanized antibody (generally, but not necessarily, comprising one or more CDRs of the antibody mBLA3), a chimeric antibody comprising variable regions derived from variable regions of the antibody mBLA3 and constant regions derived from constant regions of a human antibody, a human antibody with one or more properties of the antibody mBLA3, any of the antibodies or polypeptides described herein linked to a therapeutic agent (such as a radioactive moiety), or any of the antibodies or polypeptides described herein linked to a prodrug-activating enzyme which converts a prodrug to an active anti-cancer drug (the antibodies or the polypeptides are administered in conjunction with the prodrug) In some embodiments, the cancer is breast, colon, lung, pancreatic, prostate, esophageal, thyroid, renal, or ovarian cancer.

In yet another aspect, the invention is a method of inhibiting growth and/or proliferation of cancerous cells in an individual by administering to the individual an effective amount of a composition comprising an antibody that binds to β6 integrin subunit. In some embodiments, the antibody is the antibody mBLA3, or any of the antibodies (including polypeptides) or polynucleotides embodiments described herein, including but not limited to the antibody mBLA3 associated with a therapeutic agent, an antibody comprising a fragment or a region of the antibody mBLA3, a humanized antibody (generally, but not necessarily, comprising one or more CDRs of the antibody mBLA3), a chimeric antibody comprising variable regions derived from variable regions of the antibody mBLA3 and constant regions derived from constant regions of a human antibody, a human antibody with one or more properties of the antibody mBLA3, any of the antibodies or polypeptides described herein linked to a therapeutic agent (such as a radioactive moiety), or any of the antibodies or polypeptides described herein linked to a prodrug-activating enzyme which converts a prodrug to an active anti-cancer drug (the antibodies or the polypeptides are administered in conjunction with the prodrug). In some embodiments, the cancer is breast, colon, lung, pancreatic, prostate, esophageal, thyroid, renal, or ovarian cancer.

In yet another aspect, the invention is a method of delaying development of metastasis in an individual with cancer comprising administering an effective amount of an antibody that binds specifically to β6 integrin subunit. In some embodiments, the antibody is the antibody mBLA3, or any of the antibodies (including polypeptides) or polynucleotides embodiments described herein, including but not limited to the antibody mBLA3 associated with a therapeutic agent, an antibody comprising a fragment or a region of the antibody mBLA3, a humanized antibody (generally, but not necessarily, comprising one or more CDRs of the antibody mBLA3), a chimeric antibody comprising variable regions derived from variable regions of the antibody mBLA3 and constant regions derived from constant regions of a human antibody, a human antibody with one or more properties of the antibody mBLA3, any of the antibodies or polypeptides described herein linked to a therapeutic agent (such as a radioactive moiety), or any of the antibodies or polypeptides described herein linked to a prodrug-activating enzyme which converts a prodrug to an active anti-cancer drug (the antibodies or the polypeptides are administered in conjunction with the prodrug). In some embodiments, the cancer is breast, colon, lung, pancreatic, prostate, esophageal, thyroid, renal, or ovarian cancer.

In another aspect, the invention is a method of delivering a therapeutic agent (such as a toxin, or a radioactive molecule), or a prodrug-activating enzyme which converts a prodrug to an active anti-cancer drug to cancerous cells in an individual by administering to the individual an effective amount of an antibody that binds specifically to β6 integrin subunit or any β6 integrin subunit binding moiety (polypeptides, including but not limited to antibodies or antibody derivatives) described herein that are linked to a therapeutic agent (such as a chemotherapeutic agent, a toxin, a radioactive moiety), or a prodrug activating enzyme which converts a prodrug to an active anti-cancer drug. A β6 integrin subunit binding moiety linked to a prodrug-activating enzyme is administered in conjunction with an effective amount of the prodrug to the individual. The β6 integrin subunit binding moiety includes, but not limited to an antibody that competitively inhibits preferential binding of mBLA3 to β6 integrin subunit, an antibody that binds preferentially to the same epitope on β6 integrin subunit as mBLA3 binds preferentially, the antibody mBLA3, an antibody comprising a fragment or a region of the antibody mBLA3, or a humanized antibody (generally, but not necessarily, comprising one or more CDRs of the antibody mBLA3), a chimeric antibody comprising variable regions derived from variable regions of the antibody mBLA3 and constant regions derived from constant regions of a human antibody, or a human antibody with one or more properties of the antibody mBLA3. In some embodiments, the cancerous cells are from breast, colon, lung, pancreatic, prostate, esophageal, thyroid, renal, or ovarian cancer.

In another aspects, the invention provides kits comprising any one or more of the compositions described herein. These kits, generally in suitable packaging and provided with appropriate instructions, are useful for any of the methods described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows live cell ELISA using mBLA3, anti-αvβ6, or αvβ3. SW480 expresses αv but no β3 or β6 subunit. SKOV3 and SKMES expresses αvβ3 but not β6. SKBR3 and HT-29 expresses αvβ6 but not β3.

FIG. 2 shows antibody competition for binding on HT-29 or SKOV3 cells. R6G9 (anti-β6), E7P6 (anti-αvβ6), and 10D5 (anti-αvβ6) were used at 10 μg/ml, and mBLA3 was used at 1 μg/ml.

FIG. 3 shows antibody competition for binding on purified integrin αvβ6. R6G9 (anti-p6), E7P6 (anti-αvβ6), and 10D5 (anti-αvβ6) were used at 10 μg/ml, and mBLA3 was used at 1 μg/ml.

FIG. 4 shows effect of mBLA3 on attachment of SKBR3 to fibronectin.

FIG. 5 is a microphotograph of immunohistochemistry that shows specific binding of mBLA3 antibody (brown color) on a moderately differentiated lung adenocarcinoma and an infiltrating ductal breast adenocarcinoma. Frozen tumor samples were embedded in OCT and sectioned at 6 μm, fixed with ethanol. Immunohistochemistry was carried out as described in Example 9 for frozen sections. Cellular nuclei were counterstained with hematoxylin (pale blue).

FIG. 6 is an in situ photograph that shows the effect of mBLA3 on HT29 human tumor xenografts from athymic mice after 13 days of treatment with saline (control, bottom) or with mBLA3 (top).

FIG. 7 shows sections of the xenographs of the human HT29 tumors shown in FIG. 6 at low and high magnification. The upper panels at low magnification show the control HT29 tumor invading the liver (arrows) and surrounding healthy liver tissue. The treated tumor is compact and well differentiated compared to the poorly differentiated aggressive cancer cells in the control.

### DETAILED DESCRIPTION OF THE INVENTION

The invention provides antibodies and polypeptides which bind to a β6 subunit of integrins and methods of making and using these antibodies and polypeptides which bind to β6 subunit of integrins. β6 integrin subunit has been shown to be present and its expression is increased in a variety of human cancers. The antibody described herein binds the β6 integrin subunit. The invention further provides methods of diagnosing and treating various human cancers associated with expression of β6 integrin subunit.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

II. Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, and any other modified configuration of the immunoglobulin molecule that comprises an antigen recognition site of the required specificity.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

“Humanized” antibodies refer to a molecule having an antigen binding site substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions; e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody, which are also termed one or more CDRs “derived from” one or more CDRs from mBLA3.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

The term “immunologically active” in reference to an epitope being or “remaining immunologically active” refers to the ability of an antibody (e.g., mBLA3) to bind to the epitope under different conditions, for example, after the epitope has been subjected to reducing and denaturing conditions.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a β6 integrin subunit epitope is an antibody that binds this β6 integrin subunit epitope with greater affinity, avidity, more readily, and/or with greater duration than it binds to other β6 integrin subunit epitopes or non-β6 integrin subunit epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the terms “mBLA3”, “antibody mBLA3” and “monoclonal antibody mBLA3” are used interchangeably to refer to immunoglobulin produced by a host cell with a deposit number of ATCC No. PTA-3775 or progeny thereof. The generation and characterization of mBLA3 is described in Examples. Different biological functions are associated with mBLA3, including, but not limited to, ability to bind to β6 integrin subunit; ability to bind to native and denatured β6 integrin subunit; ability to bind to a β6 integrin subunit epitope which is disulfide bond dependent; ability to inhibit growth of cancerous cells expressing β6 integrin subunit, such as colon cancer cells; ability to delay development of metastasis in an individual with cancerous cells expressing β6 integrin subunit; ability to deliver a therapeutic agent, such as a toxin, a radioactive compound or moiety, or a prodrug-activating enzyme which converts a prodrug to an active anti-cancer drug, to cancerous cells expressing β6 integrin subunit. As discussed herein, polypeptides (including antibodies) of the invention may have any one or more of these characteristics.

A “mBLA3 equivalent antibody” or “mBLA3 equivalent polypeptide” refers to an antibody or a polypeptide having one or more biological functions associated with mBLA3, such as, for example binding specificity.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

A “variable region” of an antibody refers to the variable region of the antibody light chain or the variable region of the antibody heavy chain, either alone or in combination.

A “constant region” of an antibody refers to the constant region of the antibody light chain or the constant region of the antibody heavy chain, either alone or in combination.

As used herein, “substantially pure” refers to material which is at least 50% pure (i.e., free from contaminants), more preferably at least 90% pure, more preferably at least 95% pure, more preferably at least 98% pure, more preferably at least 99% pure, or greater, pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

An “effective amount” of an antibody, drug, or pharmaceutical composition is an amount sufficient to effect beneficial or desired results including clinical results such as shrinking the size of the tumor (in the cancer context, for example, breast or prostate cancer), retardation of cancerous cell growth, decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival of individuals. An effective amount can be administered in one or more administrations. For purposes of this invention, an effective amount of drug, compound, or pharmaceutical composition is an amount sufficient to reduce the proliferation of (or destroy) cancerous cells and to reduce and/or delay the development, or growth, of metastases of cancerous cells, either directly or indirectly. As is understood in the cancer clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality, such as administration of a prodrug to an individual in addition to administration of a β6 integrin subunit binding moiety linked to a prodrug-activating enzyme which converts the prodrug to an active anti-cancer drug to the same individual. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of metastasis” means to defer, hinder, slow, retard, stabilize, and/or postpone development of metastasis. This delay can be of varying lengths of time, depending on the history of the cancer and/or individual being treated. As is evident to one skilled in the art, a sufficient or significant delay can, in effect, encompass prevention, in that the individual does not develop the metastasis.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

An “individual” is a vertebrate, preferably a mammal, more preferably a human. Mammals include, but are not limited to, farm animals, sport animals, pets (such as cats, dogs, horses), primates, mice and rats.

“Toxin” or “cytotoxin” refers to any substance which effects an adverse response within a cell. For example, a toxin directed to a cancerous cell would have an adverse, sometimes deleterious effect, on the cancerous cell. Examples of toxins include, but are not limited to, radioisotopes, calicheamicin, and maytansinoids.

As used herein, “agent” refers to a biological, pharmaceutical , or chemical compound. Non-limiting examples include simple or complex organic or inorganic molecule, a peptide, a protein, an oligonucleotide, an antibody, an antibody derivative, or antibody fragment. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

As used herein, a “therapeutic agent” means any agent useful for therapy (here, generally in the cancer context) including anti-tumor drugs, toxins or cytotoxins, cytotoxin agents, and radioactive agents.

The term “prodrug” refers to a precursor or derivative form of a pharmaceutically active substance that is less cytotoxic to tumor cells compared to the parent drug and is capable of being enzymatically activated or converted into the more active form.

“Active immune response” refers to the development of, and on-going production of, antibodies in vivo directed against an antigen, in response to the administration of the antigen, or DNA vectors coding for that antigen, to the host mammal by intravenous, intramuscular, subcutaneous, or other mode of administration with or without an adjuvant. Active immune response can also include other aspects of the immune response, such as a cellular immune response.

III. Compositions and Methods of Making the Compositions

The invention encompasses the antibodies (including mBLA3, and mBLA3 equivalent antibodies or polypeptides (including antibodies which competitively inhibits preferential binding of mBLA3 to β6 integrin subunit, or in some embodiments, the antibodies bind to the same epitope on β6 integrin subunit as mBLA3 preferentially binds)). This invention further encompasses compositions, including pharmaceutical compositions, comprising antibodies, polypeptides and proteins described herein that bind to β6 integrin subunit, and polynucleotides comprising sequences encoding antibodies, polypeptides and proteins that bind to β6 integrin subunit. As used herein, compositions comprise one or more antibodies, polypeptides and/or proteins that bind to β6 integrin subunit, and/or one or more polynucleotides comprising sequences encoding one or more antibodies, polypeptides and proteins that bind to β6 integrin subunit. These compositions may further comprise suitable excipients, such as pharmaceutically acceptable excipients including buffers, which are well known in the art.

The antibodies, polypeptides and proteins of this invention are further identified and characterized by any (one or more) of the following criteria: (a) ability to bind to β6 integrin subunit; (b) ability to competitively inhibits preferential binding of mBLA3 to β6 integrin subunit, including the ability to preferentially bind to the β6 integrin subunit epitope to which mBLA3 preferentially binds, which is disulfide bond dependent and independent of denaturation of the antigen; (c) ability to bind β6 integrin subunit on cancer cells, such as breast, colon, lung, pancreatic, prostate, esophageal, thyroid, or renal cancer cells; (d) ability to inhibit growth and/or proliferation of cancerous cells expressing β6 integrin subunit, such as colon cancer cells; (e) ability to delay development of metastasis in an individual with cancerous cells expressing β6 integrin subunit; (f) ability to deliver a therapeutic agent, such as a toxin, a radioactive moiety, or a prodrug-activating enzyme which converts a prodrug to an active anti-cancer drug to cancerous cells expressing β6 integrin subunit.

In some embodiments, the antibody of the invention is an antibody mBLA3 that is produced by a host cell with a deposit number of ATCC No. PTA-3775 or progeny thereof. The present invention also encompasses various formulations of mBLA3 and equivalent antibodies or polypeptide fragments (e.g., Fab, Fab′, F(ab′)₂, Fv, Fc, etc.), chimeric antibodies, single chain (ScFv), mutants thereof, fusion proteins comprising an antibody portion, humanized antibodies, and any other modified configuration of mBLA3 that comprises an antigen (β6 integrin subunit), recognition site of the required specificity. The invention also provides human antibodies displaying one or more of the biological characteristics of mBLA3. The equivalent antibodies of mBLA3 (including humanized antibodies and human antibodies), polypeptide fragments of mBLA3, and polypeptides comprising any of these fragments are identified and characterized by any (one or more) of the six criteria described above.

In some embodiments, the antibodies, polypeptides and proteins of the invention that bind to β6 integrin subunit are antibodies, polypeptides and proteins that competitively inhibit preferential binding of mBLA3 to β6 integrin subunit. In some embodiments, the antibodies, the polypeptides and the proteins preferentially bind to the same epitope on β6 integrin subunit as the antibody mBLA3 preferentially binds.

Accordingly, the invention provides any of the following (or compositions, including pharmaceutical compositions, comprising any of the following): (a) antibody mBLA3 produced by the host cell with a deposit number of ATCC No. PTA-3775 or its progeny; (b) a humanized form of antibody mBLA3; (c) an antibody comprising one or more of the light chain and/or heavy chain variable regions of antibody mBLA3; (d) a chimeric antibody comprising variable regions homologous or derived from variable regions of a heavy chain and a light chain of antibody mBLA3, and constant regions homologous or derived from constant regions of a heavy chain and a light chain of a human antibody; (e) an antibody comprising one or more of the light chain and/or heavy chain CDRs (at least one, two, three, four, five, or six) of mBLA3; (f) an antibody comprising a heavy and/or a light chain of mBLA3; (g) a human antibody that is equivalent to mBLA3. A humanized form of the antibody may or may not have CDRs identical to mBLA3, or antibody produced by the host cell with a deposit number of ATCC No. PTA-3775. Determination of CDR regions is well within the skill of the art. In some embodiments, the invention provides an antibody which comprises at least one CDR that is substantially homologous to at least one CDR, at least two, at least three, at least four, at least 5 CDRs of mBLA3 (or, in some embodiments substantially homologous to all 6 CDRs of mBLA3, or derived from mBLA3), or antibody produced by the host cell with a deposit number of ATCC No. PTA-3775. Other embodiments include antibodies which have at least two, three, four, five, or six CDR(s) that are substantially homologous to at least two, three, four, five or six CDRs of mBLA3 or derived from mBLA3, or antibody produced by the host cell with a deposit number of ATCC No. PTA-3775. It is understood that, for purposes of this invention, binding specificity and/or overall activity (which may be in terms of reducing the growth and/or proliferation of cancerous cells, inducing apoptotic cell death in the cancer cell, delaying the development of metastasis, and/or treating palliatively) is generally retained, although the extent of activity may vary compared to mBLA3 (may be greater or lesser). The invention also provides methods of making any of these antibodies. Methods of making antibodies are known in the art and are described herein.

The invention also provides polypeptides comprising an amino acid sequence of the antibodies of the invention, such as mBLA3. In some embodiments, the polypeptide comprises one or more of the light chain and/or heavy chain variable regions of the antibody mBLA3. In some embodiments, the polypeptide comprises one or more of the light chain and/or heavy chain CDRs of mBLA3. In some embodiments, the polypeptide comprises three CDRs of the light chain and/or heavy chain of mBLA3. In some embodiments, the polypeptide comprises an amino acid sequence of mBLA3 that has any of the following: at least 5 contiguous amino acids of a sequence of mBLA3, at least 8 contiguous amino acids, at least about 10 contiguous amino acids, at least about 15 contiguous amino acids, at least about 20 contiguous amino acids, at least about 25 contiguous amino acids, at least about 30 contiguous amino acids, wherein at least 3 of the amino acids are from a variable region of mBLA3. In one embodiment, the variable region is from a light chain of mBLA3. In another embodiment, the variable region is from a heavy chain of mBLA3. In another embodiment, the 5 (or more) contiguous amino acids are from a complementarity determining region (CDR) of mBLA3.

Antibodies may be polyclonal (e.g., not homogeneous) or monoclonal. Methods of making monoclonal antibodies are known in the art. One method which may be employed is the method of Kohler and Milstein, Nature 256:495-497 (1975) or a modification thereof. In general, a mouse or rat is used for immunization but other animals may also be used. The immunogen can be, but is not limited to, primary cells, cultured cell lines, cancerous cells, nucleic acids, tissue, or peptides. In one embodiment, human fetal bladder cells are used. Methods for isolating and culturing human fetal bladder cells are detailed in Example 1. Cells used for immunogen, for example, human fetal bladder cells, may be cultured for a period of time (at least 24 hours) prior to their use as an immunogen. Cells (e.g., human fetal bladder cells) may be used as immunogens by themselves or in combination with a non-denaturing adjuvant, such as Ribi. In general, cells (e.g., human fetal bladder cells) should be kept intact and preferably viable when used as immunogens. Intact cells may allow antigens to be detected better by the immunized animal than ruptured cells. Use of denaturing or harsh adjuvants, e.g., Freud's adjuvant, may rupture the human fetal bladder cells and therefore is discouraged. The immunogen may be administered multiple times at periodic intervals such as, bi-weekly, or weekly, or may be administered in such a way as to maintain viability in the animal (e.g., in a tissue recombinant). Example 2 describes methods used to generate mBLA3 and may be used to generate other monoclonal antibodies which bind to β6 integrin subunit.

In one embodiment, monoclonal antibodies which bind to β6 integrin subunit are obtained by using host cells which express or overexpress β6 integrin subunit or β6 integrin subunit expressing cancer cells as an immunogen. In another embodiment, full length β6 integrin subunit or any fragments of β6 integrin subunit are used as immunogen.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells maybe screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies which bind specifically to the immunogen (e.g., surface of the HFB cells, surface of cancer cell lines, fetal bladder sections, etc.) using FACS or immunohistochemistry (IHC screening). The selected monoclonal antibody-secreting hybridomas are then cultured either in vitro (e.g., in tissue culture bottles or hollow fiber reactors), or in vivo (e.g., as ascites in mice). Examples provide further details about the methods utilized to obtain and screen an antibody mBLA3. Methods of culturing hybridoma under conditions to generate the antibody mBLA3, and purifying the antibody are known in the art and are further detailed in Examples 2 and 3.

Monoclonal antibody-secreting hybridomas described above can be selected for producing antibodies that bind preferentially to the same epitope on β6 integrin subunit that the antibody mBLA3 preferentially binds. Methods of selecting such antibody are known in the art. For example, binding competition assays can be used to determine whether an antibody binds to the same epitope as mBLA3. An antibody's competition with mBLA3 for binding to β6 integrin subunit indicates that the antibody binds preferentially to the epitope that mBLA3 binds. Binding competition assays are well known in the art and one method is detailed in Example 7. Another indication that an antibody preferentially binds to the epitope that mBLA3 preferentially binds is that the antibody binds to both native and denatured β6 integrin subunit and the epitope that the antibody binds to is disulfide bond dependent. Polypeptides that bind preferentially to the epitope on β6 integrin subunit that the antibody mBLA3 binds preferentially can also be tested and identified using similar methods.

As another alternative to the cell fusion technique, EBV immortalized B cells may be used to produce monoclonal antibodies of the subject invention. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

In another alternative, the antibodies can be made recombinantly. Methods for making recombinant antibodies are well known in the art. Monoclonal antibody mBLA3 and any other equivalent antibodies can be sequenced and produced recombinantly in vitro. In one embodiment, mBLA3 is sequenced and the polynucleotide sequence is then cloned into a vector for expression or propagation. The sequence encoding the antibody of interest may be maintained in a vector in a host cell and the host cell can then be expanded and frozen for future use. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. (1994) 12:433-455.

The antibody mBLA3 or any other antibodies or protein of interest may be subjected to sequencing by Edman degradation, which is well-known to those of skill in the art. The peptide information generated from mass spectrometry or Edman degradation can be used to design probes or primers that are used to clone the protein of interest.

An alternative method of cloning the protein of interest is by “panning” using β6 integrin subunit for cells expressing the antibody or protein of interest. The “panning” procedure is conducted by obtaining a cDNA library from tissues or cells that express the antibody or protein of interest, over-expressing the cDNAs in a second cell type, and screening the transfected cells of the second cell type for a specific binding to β6 integrin subunit. Detailed descriptions of the methods used in cloning mammalian genes coding for cell surface proteins by “panning” can be found in the art. See, for example, Aruffo, A. and Seed, B. Proc. Natl. Acad. Sci. USA, 84, 8573-8577 (1987) and Stephan, J. et al., Endocrinology 140: 5841-5854 (1999).

cDNAs can be obtained by reverse transcribing the mRNAs from a particular cell type according to standard methods in the art. Specifically, mRNA can be isolated using various lytic enzymes or chemical solutions according to the procedures set forth in Sambrook, et al. supra or extracted by commercially available nucleic-acid-binding resins following the accompanying instructions provided by manufacturers (e.g., Qiagen, Invitrogen, Promega). The synthesized cDNAs are then introduced into an expression vector to produce the antibody or protein of interest in cells of a second type. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, and cosmids.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. Preferably, the host cells express the cDNAs at a level of about 5 fold higher, more preferably 10 fold higher, even more preferably 20 fold higher than that of the corresponding endogenous antibody or protein of interest, if present, in the host cells. Screening the host cells for a specific binding to β6 integrin subunit is effected by an immunoassay or FACS. A cell overexpressed the antibody or protein of interest can be identified.

The invention includes polypeptides comprising an amino acid sequence of the antibodies of this invention, such as mBLA3. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, a mBLA3 polypeptide could be produced by an automated polypeptide synthesizer employing the solid phase method.

The invention also encompasses single chain variable region fragments (“scFv”) of antibodies of this invention, such as mBLA3. Single chain variable region fragments are made by linking light and/or heavy chain variable regions by using a short linking peptide. Bird et al. (1988) Science 242: 423-426. An example of a linking peptide is (GGGGS)₃ (SEQ ID NO:1), which bridges approximately 3.5 nm between the carboxy terminus of one variable region and the amino terminus of the other variable region. Linkers of other sequences have been designed and used. Bird et al. (1988). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention includes modifications to antibodies, such as antibody mBLA3, including functionally equivalent antibodies and polypeptides of mBLA3 which do not significantly affect their properties and variants which have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tryosine. These polypeptides also include glycosylated and nonglycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified mBLA3 polypeptides are made using established procedures in the art and can be screened using standard assays known in the art, some of which are described below and in the Examples.

The invention also encompasses fusion proteins comprising one or more fragments or regions from the antibodies of this invention, such as mBLA3. In one embodiment, a fusion polypeptide is provided that comprises at least 10 contiguous amino acids of variable light chain region and at least 10 amino acids of variable heavy chain region. In another embodiment, the fusion polypeptide contains a heterologous immunoglobulin constant region. In another embodiment, the fusion polypeptide contains a light chain variable region and a heavy chain variable region of mBLA3. For purposes of this invention, a mBLA3 fusion protein contains one or more mBLA3 polypeptides and another amino acid sequence to which it is not attached in the native molecule, for example, a heterologous sequence or a homologous sequence from another region. A mBLA3 polypeptide can be created by methods known in the art, for example, synthetically or recombinantly.

In another embodiment, mBLA3 chimeras are provided in which the heavy and/or light chains are fusion proteins. In some embodiments, the constant domain of the chains is from one particular species and/or class, and the variable domains are from a different species and/or class. For instance, a chimeric antibody (in some embodiments) is one in which the constant regions are derived from human origin, and the variable regions are homologous or derived from mBLA3 (i.e., murine). Also embodied within the invention is an antibody with a humanized variable region, in which (in some embodiments) the CDR regions comprise mBLA3 amino acid sequences, while the framework regions are derived from human sequences. Other forms of humanized antibodies are known in the art and described herein. Also embodied are functional fragments of chimeras. An example is a humanized Fab fragment, which contains a human hinge region, a human first constant region, a human kappa light or heavy chain constant region, and the variable region of light and/or heavy chain from mBLA3. The humanized mBLA3 Fab fragments can in turn be made to form Fab dimers. Typically, the mBLA3 fusion proteins and mBLA3 chimeras of this invention are made by preparing an expressing a polynucleotide encoding them using recombinant methods described herein, although they may also be prepared by other means known in the art, including, for example, chemical synthesis. See, for example, U.S. Pat. Nos. 5,807,715; 4,816,567; and 6,331,415.

The invention also encompasses humanized antibodies. The polynucleotide sequence of an antibody, such as mBLA3 or other equivalent antibodies may be used for genetic manipulation to generate a “humanized” antibody, or to improve the affinity, or other characteristics of the antibody. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. For example, the constant region may be engineered to more resemble human constant regions to avoid immune response if the antibody is used in clinical trials and treatments in humans. See, for example, U.S. Pat. Nos. 5,997,867 and 5,866,692.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; 6,350,861; and PCT WO 01/27160.

In yet another alternative, fully human antibodies may be obtained by using commercially available mice which have been engineered to express specific human immunoglobulin proteins. Transgenic animals which are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

This invention also provides mBLA3 or mBLA3 equivalent antibodies or polypeptides conjugated (for example, linked) to a therapeutic agent, such as a radioactive moiety, a toxin (e.g., calicheamicin), or a chemotherapeutic molecule, a prodrug-activating enzyme which converts a prodrug to an active anti-cancer drug, or to liposomes or other vesicles containing chemotherapeutic compounds (or compositions comprising these antibodies or polypeptides). The compositions, when administered to an individual, can target these agents to a cancer cell expressing β6 integrin subunit recognized by the antibody or polypeptide(s) and thus can, for example, eliminate (or reduce the number of) cancerous cells and/or suppress proliferation and/or growth of cancerous cells. For simplicity, reference will be made generally to mBLA3 or antibodies with the understanding that these methods apply to any of the β6 integrin subunit binding embodiments described herein. These, conjugation generally refers to linking these components as described herein. The linking (which is generally fixing these components in proximate association at least for administration) can be achieved in any number of ways, as described below.

A radioactive moiety or molecule of this invention includes any radioisotope which is effective in destroying a cancerous cell. Examples include, but not limited to, cobalt-60 and X-rays. Additionally, naturally occurring radioactive elements such as uranium, radium, and thorium which typically represent mixtures of radioisotopes, are suitable examples of a radioactive molecule.

A toxin of the invention include, but not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The antibodies or polypeptides of the invention can be conjugated (linked) to a radioactive moiety or molecule, a toxin, or other therapeutic agents, a prodrug-activating enzyme which converts a prodrug to an active anti-cancer drug, or to liposomes or other vesicles containing therapeutic agents covalently or non-covalently, directly or indirectly. The antibody may be linked to the radioactive molecule, the toxin, the therapeutic molecule, or a prodrug-activating enzyme at any location along the antibody so long as the antibody is able to bind its target β6 integrin subunit.

A toxin or a therapeutic agent may be coupled (e.g., covalently bonded) to a suitable monoclonal antibody either directly or indirectly (e.g., via a linker group, or, alternatively, via a linking molecule with appropriate attachment sites, such as a platform molecule as described in U.S. Pat. No. 5,552,391). The toxin and therapeutic agent of the present invention can be coupled directly to the particular targeting proteins using methods known in the art. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies or polypeptides can also be linked to a therapeutic agent via a microcarrier. Microcarrier refers to a biodegradable or a non-biodegradable particle which is insoluble in water and which has a size of less than about 150, 120 or 100 μm in size, more commonly less than about 50-60 μm, preferably less than about 10, 5, 2.5, 2 or 1.5 μm. Microcarriers include “nanocarriers”, which are microcarriers have a size of less than about 1 μm, preferably less than about 500 num. Such particles are known in the art. Solid phase microcarriers may be particles formed from biocompatible naturally occurring polymers, synthetic polymers or synthetic copolymers, which may include or exclude microcarriers formed from agarose or cross-linked agarose, as well as other biodegradable materials known in the art. Biodegradable solid phase microcarriers may be formed from polymers which are degradable (e.g., poly(lactic acid), poly(glycolic acid) and copolymers thereof) or erodible (e.g., poly(ortho esters such as 3,9-diethylidene-2,4,8,10-tetraoxaspiro[5.5]undecane (DETOSU) or poly(anhydrides), such as poly(anhydrides) of sebacic acid) under mammalian physiological conditions. Microcarriers may also be liquid phase (e.g., oil or lipid based), such liposomes, iscoms (immune-stimulating complexes, which are stable complexes of cholesterol, and phospholipid, adjuvant-active saponin) without antigen, or droplets or micelles found in oil-in-water or water-in-oil emulsions, provided the liquid phase microcarriers are biodegradable. Biodegradable liquid phase microcarriers typically incorporate a biodegradable oil, a number of which are known in the art, including squalene and vegetable oils. Microcarriers are typically spherical in shape, but microcarriers which deviate from spherical shape are also acceptable (e.g., elipsoid, rod-shaped, etc.). Due to their insoluble nature (with respect to water), microcarriers are filterable from water and water-based (aqueous) solutions.

The antibody or polypeptide conjugates of the present invention may include a bifunctional linker which contains both a group capable of coupling to a toxic agent or therapeutic agent and a group capable of coupling to the antibody. A linker can function as a spacer to distance an antibody from an agent in order to avoid interference with binding capabilities. A linker can be cleavable or non-cleavable. A linker can also serve to increase the chemical reactivity of a substituent on an agent or an antibody, and thus increase the coupling efficiency. An increase in chemical reactivity may also facilitate the use of agents, or functional groups on agents, which otherwise would not be possible. The bifunctional linker can be coupled to the antibody by means which are known in the art. For example, a linker containing an active ester moiety, such as an N-hydroxysuccinimide ester, can be used for coupling to lysine residues in the antibody via an amide linkage. In another example, a linker containing a nucleophilic amine or hydrazine residue can be coupled to aldehyde groups produced by glycolytic oxidation of antibody carbohydrate residues. In addition to these direct methods of coupling, the linker can be indirectly coupled to the antibody by means of an intermediate carrier such as an aminodextran. In these embodiments the modified linkage is via either lysine, carbohydrate, or an intermediate carrier. In one embodiment, the linker is coupled site-selectively to free thiol residues in the protein. Moieties which are suitable for selective coupling to thiol groups on proteins are well known in the art. Examples include disulfide compounds, α-halocarbonyl and α-halocarboxyl compounds, and maleimides. When a nucleophilic amine function is present in the same molecule as an α-halo carbonyl or carboxyl group the potential exists for cyclization to occur via intramolecular alkylation of the amine. Methods to prevent this problem are well known to one of ordinary skill in the art, for example by preparation of molecules in which the amine and α-halo functions are separated by inflexible groups, such as aryl groups or trans-alkenes, that make the undesired cyclization stereochemically disfavored. See, for example, U.S. Pat. No. 6,441,163 for preparation of conjugates of maytansinoids and antibody via a disulfide moiety.

An antibody (or polypeptide) of this invention may be conjugated (linked) to a radioactive moiety or molecule by any method known to the art. For a discussion of methods for radiolabeling antibody see “Cancer Therapy with Monoclonal AntibodiesT”, D. M. Goldenberg ed. (CRC Press, Boca Raton, 1995).

The antibodies (or polypeptides) of the invention may be linked to an agent (including a prodrug-activating enzyme) which converts a prodrug to an active anti-cancer drug. For example, the antibodies (or polypeptides) of this invention may be used in Antibody Dependent Enzyme Mediated Prodrug Therapy (ADEPT) by conjugating (linking) the antibody to a prodrug-activating enzyme which converts a prodrug (e.g., a peptidyl chemotherapeutic agent, see WO81/01 145) to an active-cancer drug. See, for example, WO 88/07378 and U.S. Pat. No. 4,975,278.

The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328:457-458 (1987); Carter, Nat. Rev. Cancer 1(2):118-29 (2001); U.S. Pat. No. 6,368,839; U.S. Pat. No. 5,658,753). Antibody-abzyme conjugates can be prepared as described herein for delivery of the abzyme to a tumor cell population.

The enzymes of this invention can be covalently bound to the antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312:604-608 (1984)).

The antibodies or polypeptides of the invention linked to a prodrug-activating enzyme can be used in conjunction with a prodrug which is converted by the enzyme into an active anti-cancer drug. See, e.g., D. E. V. Wilman, “Prodrugs In Cancer Chemotherapy,” Biochemical Society Transactions, 14, pp. 375-382 (615th Meeting, Belfast 1986) and V. J. Stella et al., “Prodrugs: A Chemical Approach To Targeted Drug Delivery,” Directed Drug Delivery, R. Borchardt et al. (ed.), pp.247-267 (Humana Press 1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta.-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted by the enzyme of the conjugate into the more active, cytotoxic free drug. Examples of cytotoxic drugs that can be derivatized into a prodrug form for use in this invention include, but are not limited to, etoposide, teniposide, adriamycin, daunomycin, carminomycin, aminopterin, dactinomycin, mitomycins, cis-platinum and cis-platinum analogues, bleomycins, esperamicins (U.S. Pat. No. 4,675,187), 5-fluorouracil, melphalan and other related nitrogen mustards.

An antibody (or polypeptide) of this invention may be linked to a labeling agent (alternatively termed “label”) such as a fluorescent molecule, a radioactive molecule or any others labels known in the art. Labels are known in the art which generally provide (either directly or indirectly) a signal.

The ability of the antibodies or polypeptides of this invention, such as ability to inhibit growth of cancerous cells expressing β6 integrin subunit, ability to delay development of metastasis in an individual with cancer expressing β6 integrin subunit, ability to deliver a therapeutic agent, such as a toxin, or a radioactive compound, or a prodrug-activating enzyme which converts a prodrug to an active anti-cancer drug to cancerous cells expressing β6 integrin subunit, may be tested using methods known in the art, some of which are described in the Examples.

The invention also provides compositions (including pharmaceutical compositions) comprising antibody mBLA3 or mBLA3 equivalent antibodies (which, as this disclosure makes clear, include all of the antibodies described herein) or polypeptides and a therapeutic agent.

IV. Methods for Screening Monoclonal Antibodies

Several methods may be used to screen monoclonal antibodies that bind to P6 integrin subunit. It is understood that “binding” refers to immunologically relevant binding, i.e., binding which is specific for the antigen (or the epitope) for which the immunoglobulin molecule is encoded. It does not refer to non-specific binding that may occur when an immunoglobulin is used at a very high concentration against a non-specific target. One method which may be employed for screening is immunohistochemistry (IHC). Standard immunohistochemical techniques are known to those of average skill in the art. See, for example, Animal Cell Culture Methods (J. P. Mather and D. Barnes, eds., Academic Press, Vol. 57, Ch. 18 and 19, pp. 314-350, 1998). Biological samples (e.g., tissues) may be obtained from biopsies, autopsies, or necropsies. To ascertain if β6 integrin subunit is present only on cancerous cells, mBLA3 may be used to detect the presence of β6 integrin subunit on tissues from individuals with cancer while other non-cancerous tissues from the individual suffering from cancer or tissues from individuals without cancer are used as a control. The tissue can be embedded in a solid or semi-solid substance which prevents damage during freezing (e.g., agarose gel or OCT) and then sectioned for staining. Cancers from different organs and at different grades can be used to screen monoclonal antibodies. Examples of tissues which may be used for screening purposes include but are not limited to ovary, breast, lung, prostate, colon, kidney, skin, thyroid, brain, heart, liver, stomach, nerve, blood vessels, bone, upper digestive tract, and pancreas. Examples of different cancer types which may be used for screening purposes include but are not limited to carcinomas, adenocarcinomas, sarcomas, adenosarcomas, lymphomas, and leukemias. 101101 In yet another alternative, cancerous cells lines such as SK-Ov-3 (ATCC #HTB 77), OVCAR-3 (ATCC #HTB 161), Caov-3 (ATCC #HTB 75), LnCap (ATCC #CRL-1740), COLO 205 (ATCC #CCL 222), A549 (ATCC #CCL 185), PANC-1 (ATCC #CRL 1469), SK-BR-3 (ATCC #HTB 30), SK-MES-1 (ATCC #HTB 58), HT-29 (HTB-38), H9 (ATCC #HTB-176), SW 480 (ATCC #CCL 228), AsPC-1 (ATCC #CRL 1682), Capan-1 (ATCC #HTB 79), CFPAC-1 (ATCC #CRL 1918), HPAF-II (ATCC #CRL-1997), HF-700T (ATCC #HTB 147), ES-2 (ATCC #CRL-1978), PC-3 (ATCC #CRL 1435), END (human fibroblast cells cultured from adult endometrium in), Du-145 (ATCC #HTB-81), Rav CA130 (proprietary lung cancer line developed at Raven Biotechnologies, Inc.), Rav9926 (proprietary pancreatic cancer cell line), SU.86.86 (ATCC CRL-1837), and ZR7-51 (ATCC #CRL-1500) and normal cells from their respective tissues may be used to screen for monoclonal antibodies which are specific for cancerous tissue. Primary, or low passage, cell cultures derived from normal tissues from different organs, including but not limited to, ovary, breast, lung, prostate, colon, kidney, skin, thyroid, aortic smooth muscle, and endothelial cells can be used as negative controls. The cancerous or non-cancerous cells can be grown on glass slides or coverslips, or on plastic surfaces, or prepared in a CellArray™, as described in WO 01/43869, and screened for the binding of antibody using IHC as described above for tissues. Alternatively, cells may be removed from the growth surface using non-proteolytic means and spun into a pellet which is then embedded and treated as tissues for IHC analysis as described above. In another alternative, single cells may be screened by incubating with the primary antibody, a secondary “reporter” antibody linked to a fluorescent molecule and then analyzed using a fluorescent activated cell sorting (FACS) machine.

Several different detection systems may be utilized to detect binding of antibodies to tissue section. Typically, immunohistochemistry involves the binding of a primary antibody to the tissue and then a secondary antibody reactive against the species from the primary antibody was generated and conjugated to a detectable marker (e.g., horseradish peroxidase, HRP, or diaminobenzedine, DAB). One alternative method that may be used is polyclonal mirror image complementary antibodies or polyMICA. PolyMICA (polyclonal Mirror Image Complementary Antibodies) technique, described by D. C. Mangham and P. G. Isaacson (Histopathology (1999) 35(2):129-33), can be used to test binding of primary antibodies (e.g., mBLA3) to normal and cancerous tissue. Several kinds of polyMICA™ Detection kits are commercially available from The Binding Site Limited (P.O. Box 4073 Birmingham B29 6AT England). Product No. HK004.D is a polyMICA™ Detection kit which uses DAB chromagen. Product No. HK004.A is a polyMICA™ Detection kit which uses AEC chromagen. Alternatively, the primary antibody may be directly labeled with the detectable marker.

The first step in IHC screening to select for an appropriate antibody is the binding of primary antibodies raised in mice (e.g., mBLA3) to one or more immunogens (e.g., cells or tissue samples). In one embodiment, the tissue sample is sections of frozen tissue from different organs. The cells or tissue samples can be either cancerous or non-cancerous.

Frozen tissues can be prepared, sectioned, with or without fixation, and IHC performed by any of a number of methods known to one familiar with the art. See, for example, Stephan et al. Dev. Biol. 212: 264-277 (1999), and Stephan et al. Endocrinology 140: 5841-54 (1999).

Monoclonal antibodies which bind to β6 integrin subunit are screened for binding to cancerous cells or tissues, but not to normal cells or tissues to the same degree, are selected. Monoclonal antibodies that bind to antigens expressed on one or more cancer types but not to normal cells are also selected. mBLA3 is an example of an antibody that binds to an antigen present on a number of different cancers, but has limited binding to normal tissues. In accordance with the Budapest Treaty, the hybridoma which produces mBLA3 has been deposited in the American Type Culture Collection (ATCC) 10801 University Blvd., Manassas Va. 20110-2209 on Oct. 10, 2001 with a Patent Deposit Designation of PTA-3775.

V. Methods of Characterizing mBLA3

Several methods are used to characterize mBLA3. One method is to identify the epitope to which it binds. Epitope mapping is commercially available from various sources, for example, Pepscan Systems (Edelhertweg 15, 8219 PH Lelystad, The Netherlands). Epitope mapping can be used to determine the sequence to which mBLA3 binds. The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with mBLA3. The epitope to which mBLA3 binds can be determined in a systematic screening by using overlapping peptides derived from the β6 integrin subunit extracellular sequence and determining binding by mBLA3.

Yet another method which can be used to characterize mBLA3 is to use competition assays with other antibodies known to bind to the same antigen, i.e., β6 integrin subunit to determine if mBLA3 binds to the same epitope as other antibodies. Examples of commercially available antibodies to β6 integrin subunit are R6G9, 10D5, and E7P6. Competition assays are well known to those of skill in the art. This procedure is detailed further in Example 7. mBLA3 can be further characterized by the tissues, type of cancer or type of tumor to which it binds.

Another method of characterizing mBLA3 is by the antigen to which it binds. mBLA3 was used in Western blots with cell lysates from various human cancers. As is known to one of skill in the art, Western blotting can involve running cell lysates and/or cell fractions on a denaturing or non-denaturing gel, transferring the proteins to nitrocellulose paper, and then probing the blot with an antibody (e.g., mBLA3) to see which proteins are bound by the antibody. This procedure is detailed further in Example 4. The band to which mBLA3 bound was isolated and further analyzed using mass spectroscopy, as described in Examples 5 and 6. The antigen to which mBLA3 binds was found to be β6 subunit of integrins, specifically in the context of αvβ6 integrin. While binding of mBLA3 is shown herein to bind to αvβ6, the invention is not limited to binding to αvβ6 integrin only. Any integrins which comprise β6 subunit can also be recognized by mBLA3 and thus, be useful for screening, diagnosis, and treatment purposes.

VI. Methods of Diagnosing Cancer using mBLA3, mBLA3 equivalent Antibodies or Polypeptides which Bind to β6 Integrin Subunit

Monoclonal antibody mBLA3 and equivalent antibodies or polypeptides derivatives of mBLA3 which bind to β6 integrin subunit made by the methods disclosed herein may be used to identify the presence or absence of cancerous cells in a variety of tissues, including but not limited to, ovary, breast, lung, prostate, colon, kidney, skin, thyroid, brain, heart, liver, stomach, nerve, blood vessels, bone, upper digestive tract, and pancreas for purposes of diagnosis. For simplicity, reference will be made generally to mBLA3. or antibodies with the understanding that these methods apply to any of the β6 integrin subunit binding embodiments described herein. Detection generally involves contacting cells with an antibody or a polypeptide described herein that binds to β6 integrin subunit and the formation of a complex between β6 integrin subunit and an antibody (e.g., mBLA3, a humanized antibody of mBLA3, a human antibody or any other β6 integrin subunit binding moiety) which binds specifically to β6 integrin subunit. The formation of such a complex can be in vitro or in vivo. Without being bound by theory, monoclonal antibody mBLA3 can bind to β6 integrin subunit through the extracellular domain of β6 subunit.

In some embodiments, methods are provided for detecting presence or absence of prostate cancerous cells by detecting β6 integrin subunit from cells. β6 integrin subunit from prostate cancerous cells can be detected using any method, including but not limited to detection of β6 integrin subunit mRNA, and detection of β6 integrin subunit protein. Any β6 integrin subunit binding moiety can be used, such as those described herein (e.g., mBLA3, mBLA3 equivalent antibodies such as those which competitively inhibits preferential binding of mBLA3 to β6 integrin subunit). As used herein, detection may include qualitative and/or quantitative detection and may include comparing the level measured to a normal prostate cell for an increased level of expression of β6 integrin subunit in cancerous cells.

In another aspect, the invention provides methods of aiding diagnosis of cancer using any antibodies or polypeptides described herein. As used herein, methods for “aiding diagnosis” means that these methods assist in making a clinical determination regarding the classification, or nature, of cancer, and may or may not be conclusive with respect to the definitive diagnosis. Accordingly, a method of aiding diagnosis of cancer can comprise the step of detecting the level of β6 integrin subunit in a biological sample from the individual and/or determining the level of β6 integrin subunit expression in the sample.

One method of using the antibodies for diagnosis is in vivo tumor imaging by linking the antibody to a labeling moiety (e.g., a fluorescent agent, a radioactive or radioopaque agent), administering the antibody to the individual and using an x-ray or other imaging machine to visualize the localization of the labeled antibody at the surface of cancer cells expressing the antigen. The antibody is administered at a concentration that promotes binding at physiological conditions. Labeling moieties are known in the art.

In other methods, the cancerous cells are removed and the tissue prepared for immunohistochemistry by methods well known in the art (e.g., embedding in a freezing compound, freezing and sectioning, with or without fixation; fixation and paraffin embedding with or without various methods of antigen retrieval and counterstaining). The monoclonal antibodies may also be used to identify cancerous cells at different stages of development. The antibodies may also be used to determine which individuals' tumors express the antigen on their surface at a pre-determined level and are thus candidates for immunotherapy using antibodies directed against said antigen.

Antibodies (or polypeptides) recognizing the antigen may also be used to create diagnostic immunoassays for detecting antigen released or secreted from living or dying cancer cells in bodily fluids, including but not limited to, blood, saliva, urine, pulmonary fluid, or ascites fluid. As discussed in further detail in the Examples, mBLA3 can bind to various forms cancer in different stages from tissues including but not limited to breast, colon, lung, pancreatic, prostate, esophageal, thyroid, and kidney. Methods of using mBLA3 for diagnostic purposes is useful both before and after any form of anti-cancer treatment, e.g., chemotherapy or radiation therapy, to determine which tumors are most likely to respond to a given treatment, prognosis for individual with cancer, tumor subtype or origin of metastatic disease, and progression of the disease or response to treatment.

VII. Methods of Using mBLA3, mBLA3 Equivalent Antibodies or Polypeptides for Therapeutic Purposes

Monoclonal antibody mBLA3 and equivalent antibodies (as well as other polypeptides embodiments of the invention) made by the methods disclosed herein may be used for therapeutic purposes in individuals with cancer in a variety of tissues, including but not limited to, ovary, breast, lung, prostate, colon, kidney, skin, thyroid, brain, heart, liver, stomach, nerve, blood vessels, bone, upper digestive tract, and pancreatic cancer. In some embodiments, the cancer is prostate (provided that the prostate cancer cells bind to the antibody or polypeptide). These therapeutic methods also apply to the linked embodiments described above. For simplicity, reference will be made generally to mBLA3 or antibodies with the understanding that these methods apply to any of β6 integrin subunit binding embodiments described herein, including but not limited to humanized antibodies and human antibodies described herein including linked embodiments. Therapy with mBLA3 can involve formation of complexes both in vitro and in vivo as described above. In one embodiment, monoclonal antibody mBLA3 can bind to and reduce the proliferation of cancerous cells (e.g., colon carcinoma cells) as shown in Examples 12 and 13. It is understood that the antibody is administered at a concentration that promotes binding at physiological (e.g., in vivo) conditions. In another embodiment, monoclonal antibody mBLA3 alone can bind to and induce apoptotic cell death in the cancer cell. In another embodiment, monoclonal antibody mBLA3 can bind to cancerous cells and delay the development of metastasis. In another embodiment, monoclonal antibody mBLA3 can bind to cancerous cells and deliver a therapeutic agent (such as a toxin, or a radioactive compound) or an agent (including but not limited to a prodrug-activating enzyme) which converts a prodrug to an active anti-cancer drug linked to mBLA3 to cancerous cells. The antibody linked to an agent (such as a prodrug-activating enzyme) is administered in conjunction with the prodrug to the individual. Generally, in these embodiments an effective amount (an amount sufficient to deliver a therapeutic agent to target cancerous cells or an amount sufficient to deliver a prodrug-activating enzyme which converts a prodrug to a sufficient amount of active anti-cancer drug) is administered to an individual. In yet another embodiments, an individual with cancer is given palliative treatment with antibodies of the invention. Palliative treatment of a cancer individual involves treating or lessening the adverse symptoms of the disease, or iatrogenic symptoms resulting from other treatments given for the disease without directly affecting the cancer progression. This includes treatments for easing of pain, nutritional support, sexual problems, psychological distress, depression, fatigue, psychiatric disorders, nausea, vomiting, etc.

This invention also provides methods of treating cancer and inhibiting growth and/or proliferation of cancer cells, including but not limited to breast, colon, lung, pancreatic, prostate, esophageal, thyroid, renal, and ovarian cancer cells using any antibody or other embodiments that bind to β6 integrin subunit. Treatment methods of the invention apply to any cancer expressing β6 integrin subunit on the cell surface. Antibodies that bind to β6 integrin subunit have been described. See, for example, U.S. Pat. No. 6,316,601. The method of testing activity of an antibody in inhibiting growth and/or proliferation of cancer cells are known in the art and are described in detail in Examples 12 and 13.

This invention also provides methods of inhibiting growth and/or proliferation of cancer cells (such as prostate, lung, breast, colon, ovarian, pancreatic, renal, ovarian cancer cells) using mBLA3 antibody or an mBLA3 equivalent antibody or polypeptide (such as an antibody that competitively inhibits preferential binding of mBLA3 to β6 integrin subunit). In some embodiments, the antibody preferentially binds to the same epitope as mBLA3 preferentially binds.

In yet another embodiment, mBLA3 or any of the β6 integrin subunit embodiments described herein can bind to β6 integrin subunit expressing cancerous cells and induces an active immune response against the cancerous cells expressing β6 integrin subunit. In some cases, the active immune response can cause the death of the cancerous cells (e.g., mBLA3 binding to cancer cells inducing apoptotic cell death), or inhibit the growth (e.g., block cells cycle progression) of the cancerous cells. In other cases, mBLA3 or any of the β6 integrin subunit antibodies described herein can bind to cancerous cells and antibody dependent cellular cytotoxicity (ADCC) can eliminate cancerous cells to which mBLA3 binds. Accordingly, the invention provides methods of stimulating an immune response comprising administering any of the compositions described herein.

In some cases, mBLA3 binding can also activate both cellular and humoral immune responses and recruit more natural killer cells or increased production of cytokines (e.g., IL-2, IFN-γ, IL-12, TNF-α, TNF-β, etc.) that further activate an individual's immune system to destroy cancerous cells. In yet another embodiment, mBLA3 can bind to cancerous cells, and macrophages or other phagocytic cell can opsonize the cancerous cells.

In some embodiments, the invention provides methods of conferring passive immunity comprising administering any of the compositions described herein.

The invention provides methods of delivering any of the compositions (including conjugates) described herein to a β6 integrated subunit expressing cell, such as a β6 integrated subunit expression cancer cells. These methods entail administering the compositions (including conjugates) described herein to an individual. The antibody mBLA3 and equivalent antibodies (as well as other polypeptides embodiments of the invention) made by the methods disclosed herein may be conjugated to or associated with a radioactive moiety or molecule, toxin (e.g., calicheamicin), chemotherapeutic molecule, a prodrug-activating enzyme which converts a prodrug to an active anti-cancer drug, liposomes or other vesicles containing chemotherapeutic compounds and administered to an individual to target these compounds to the cancer cell containing the antigen recognized by the antibody and thus eliminate cancerous cells. In yet another embodiment, an individual with cancer is given palliative treatment with mBLA3. In yet another embodiment, the antibody can be employed as adjuvant therapy at the time of the surgical removal of a cancer expressing the antigen in order to delay the development of metastasis. The antibody can also be administered before surgery (neoadjuvant therapy) in an individual with a tumor expressing the antigen in order to decrease the size of the tumor and thus enable or simplify surgery, spare tissue during surgery, and/or decrease the resulting disfigurement.

Various formulations of mBLA3 and equivalent antibodies or fragments (e.g., Fab, Fab′, F(ab′)₂, Fv, Fc, etc.), such as chimeric antibodies, single chain (ScFv), mutants thereof, fusion proteins comprising an antibody portion, humanized antibodies, and any other modified configuration of mBLA3 that comprises a β6 integrin subunit recognition site of the required specificity, thereof may be used for administration. In some embodiments, mBLA3 antibodies or various formulations of mBLA3 thereof may be administered neat. In other embodiments, mBLA3 or various formulations of mBLA3 (including any composition embodiment described herein) thereof and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.), although other forms of administration (e.g., oral, mucosal, etc) can be also used. Accordingly, mBLA3 antibody and equivalents thereof are preferably combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of at least about 100 ug/kg body weight, at least about 250 ug/kg body weight, at least about 750 ug/kg body weight, at least about 3 mg /kg body weight, at least about 5 mg /kg body weight, at least about 10 mg/kg body weight is administered.

Empirical considerations, such as the half-life, generally will contribute to the determination of the dosage. Antibodies which are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is based on reducing the number of cancerous cells, maintaining the reduction of cancerous cells, reducing the proliferation of cancerous cells, or delaying the development of metastasis. The presence of cancerous cells can be identified by any number of methods known to one of skill in the art or discussed herein (e.g., detection by immunohistochemistry or flow cytometry of biopsies or biological samples). Alternatively, sustained continuous release formulations of mBLA3 antibodies may be appropriate. Various formulations and devices for achieving sustained release are known in the art.

In one embodiment, dosages for mBLA3 antibodies may be determined empirically in individuals who have been given one or more administration(s). Individuals are given incremental dosages of mBLA3. To assess efficacy of mBLA3 or other equivalent antibody, a marker of the specific cancer disease state can be followed. These include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer), a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In some embodiments, more than one antibody may be present. The antibodies can be monoclonal or polyclonal. Such compositions may contain at least one, at least two, at least three, at least four, at least five different antibodies that are reactive against carcinomas, adenocarcinomas, sarcomas, or adenosarcomas. mBLA3 antibody can be admixed with one or more antibodies reactive against carcinomas, adenocarcinomas, sarcomas, or adenosarcomas in organs including but not limited to ovary, breast, lung, prostate, colon, kidney, skin, thyroid, bone, upper digestive tract, and pancreas. A mixture of antibodies, as they are often denoted in the art, may be particularly useful in treating a broader range of population of individuals.

Assessment of disease is performed using standard methods in the arts, such as imaging methods and monitoring appropriate marker(s).

VIII. Kits Comprising Antibodies and Polypeptides of the Invention which bind to β6 Integrin Subunit

The invention also provides kits comprising antibodies or any of the compositions described herein which bind to β6 integrin subunit for use in diagnosis and/or therapy. Accordingly, the kits comprise an antibody which can bind to β6 integrin subunit preferentially and/or form a complex with β6 integrin subunit (useful, for example, for detecting breast, colon, lung, ovarian, pancreatic, prostate, esophageal, thyroid, renal, or ovarian cancerous cells). In some embodiments, the kits comprise antibody mBLA3 or an antibody that competitively inhibits preferential binding of mBLA3 to β6 integrin subunit. In some embodiments, the kits comprise antibody mBLA3 or an antibody that competitively inhibits preferential binding of mBLA3 to β6 integrin subunit linked to a therapeutic agent, a prodrug-activating enzyme, or a labeling agent. These kits may further include instruction and/or reagents for linking the antibody or any antibody or polypeptide embodiments described herein to the therapeutic agent(s), the prodrug-activating enzyme, or the labeling agent(s). In some aspects, the binding of an antibody (e.g., monoclonal, polyclonal, human, humanized, etc.) to β6 integrin subunit is used for diagnosing cancer in an individual, for example, kits for detecting presence or absence of cancerous cells, and kits for detecting presence or absence of breast, colon, lung, ovarian, pancreatic, prostate, esophageal, thyroid, renal, or ovarian cancerous cells. In other aspects, the kits may be used, for example, to treat an individual with cancer or a family history of cancer. Kits for treating individual with cancer include but not limited to kits for inhibiting growth and/or proliferation of cancer cells, such as colon cancer cells, and/or for delivering a therapeutic agent or a prodrug-activating enzyme to cancerous cells. The kits of this invention are in suitable packaging, and may optionally provide additional components such as, a prodrug to be used in conjunction with the antibody of the invention, and buffers and instructions for determining binding to β6 integrin subunit, such as capture reagents, developing reagents, labels, reacting surfaces, means for detection, control samples, and interpretive information. The instructions may be for any measurement of antigen binding, including, but not limited to, those assays described herein. In other embodiments, the instructions may be for any of the methods described herein, including: instructions for inhibiting grow and/or proliferation of cancerous cells such as colon cancerous cells, for delivering a therapeutic agent or a prodrug-activating enzyme to cancerous cells (including instruction for use of a prodrug). In some embodiments, reagents described above are supplied such that multiple measurements may be made, such as allowing for measurements in the same individual over time or multiple individuals. Any appropriate means for detecting binding of the antibodies may be employed (and provided in the kits) such as a labeled anti-human antibody, wherein the label may be an enzyme, fluorophore, chemiluminescent material radioisotope or coenzyme. Generally, the label used will be an enzyme.

The following examples are provided to illustrate, but not to limit, the invention.

### EXAMPLES

### Example 1

### Preparation of Human Fetal Bladder Cells as an Immunogen

Human fetal bladders of gestational age between 14 to 21 weeks were obtained from Advanced Bioscience Research at Alameda County, California. Bladders were procured and shipped to the lab in tissue culture medium on wet ice. Immediately upon arrival, the bladders were washed three times with 20 ml cool PBS. Under the dissection microscope, bladders were cleaned of extra tissue around bladder, and washed two more times with cool PBS.

The bladders were minced into 1 mm cube with a razor blade in a 100 mm dry culture dish. Ten ml Opti-MEM medium (GIBCO BRL Cat. No. 22600) was added. The tissue pieces were transferred into a 15 ml centrifuge tube by a 5 ml pipette, which was pre-coated with 5% BSA in PBS. The tissue pieces were then centrifuged at 1000×g for 5 minutes. The pellet was resuspended in 6 ml Opti-MEM medium. The tissue was cultured in a 6 well-plate with 3 ml Opti-MEM medium, which contained following growth factors (final concentration in each milliliter medium): insulin 10 μg/ml, transferrin 10 μg/ml, vitamin E 5 μg/ml, aprotinin 25 μg/ml, progesterone 3 ng/ml, KGF 10 ng/ml, heregulin (HRG) 5 nM, gentamycin 100 μg/ml (added in the first two days cell culture). Trypsin (GIBCO BRL Cat. No. 25300-054) at final concentration of 0.05% was added to get rid of the fibroblast cell three days before injection.

Under these culture conditions, the human fetal bladder cells (HFB) attached to the plastic of the tissue culture container and grew as a monolayer. Cultures were passaged by first trypsinizing the cells to detach them from the tissue culture container and then re-plating the cells in the same culture medium at a 1 to 5 split ratio every 5 to 7 days. The cells were washed with Opti-MEM and grown in serum free medium as described above for a minimum of 24 hour prior to harvesting for injections.

To harvest the cells, the cells were rinsed once with calcium and magnesium free Hanks saline solution, incubated in 0.02% EDTA in Hanks saline solution at 37° C. for 15 minutes. The cells were detached from the culture surface by gentle tapping. The cell suspension was precipitated by centrifuge at 1000 rpm for 10 minutes. The supernatant was removed and cells were resuspended in serum free medium (Opti-MEM) containing appropriate non-denaturing adjuvant.

### Example 2

### Generation of Monoclonal Antibodies

HFB cells between 11 and 16 days of culture were used to generate monoclonal antibodies. Approximately 10⁶ HFB cells per mouse were injected into Balb/c mice via foot-pad, once a week. Non-denaturing adjuvants, (e.g., Ribi) were used. After 6 weeks of weekly injection, a drop of blood were drawn from the tail of each immunized animal to test the titer of antibodies against HFB using FACS analysis. When the titer reached at least 1:2000, the mice were sacrificed in a CO₂ chamber followed by cervical dislocation. Lymph nodes were harvested for hybridoma preparation.

Lymphocytes from mice with the highest titer were fused with the mouse myeloma line X63-Ag8.653 using 35% polyethylene glycol 4000. On day 10 following the fusion, the hybridoma supernatants were screened for the presence of HFB- specific monoclonal antibodies by fluorescence activated cell sorting (FACS). Conditioned medium from each hybridoma was incubated for 30 minutes with an aliquot of HFB cells. After incubation, the cell samples were washed, resuspended in 0.1 ml diluent and incubated with 1 μg/ml of FITC conjugated F(ab′)2 fragment of goat anti-mouse IgG for 30 min at 4° C. The cells were washed, resuspended in 0.5 ml FACS diluent and analyzed using a FACScan cell sorter (Becton Dickinson; San Jose, Calif.). Hybridoma clones were selected for further expansion, cloning, and characterization based on their binding to the surface of HFB cells as assessed by FACS. One hundred hybridoma clones were found positive in this primary screen.

The positive hybridomas were further screened for their reaction to normal tissue sections. Hybridoma clones stained positive on bladder and prostate tissue sections and negative on kidney and skin tissue sections were collected. A hybridoma making a monoclonal antibody designated mBLA3 was selected. In accordance with the Budapest Treaty, the hybridoma which produces mBLA3 was deposited in the American Type Culture Collection (ATCC)P.O.Box 1549 Manassas, Va. 20108 on Oct. 10, 2001 with a Patent Deposit Designation of PTA-3775.

### Example 3

### Purification of mBLA3

Monoclonal antibodies were purified from tissue culture supernatant using protein-G affinity chromatography. The following materials were used for the antibody purification process: hybridoma tissue culture supernatant, Immunopure (G) IgG binding buffer (Pierce #21011 Rockford, Ill.), Immunopure IgG Elution Buffer (Pierce #21009), concentrated HCl (for adjusting pH), Corning 1 liter PES (polyether sulfone), 0.22 μm filter (Corning #431098, Corning, N.Y.), Amersham Pharmacia GradiFrac System (Amersham Pharmacia, Piscataway, N.J.), Protein-G Sepharose 4 Fast Flow (AmershamPharmacia #17-0618-02), Stripping buffer which is 3M KSCN/50 mM Tris pH 7.8, and PBS (phosphate buffered saline) 3M Tris pH 9.0.

To purify the mBLA3 antibody, the volume of supernatant was measured and an equal volume of binding buffer was added to the supernatant. The mixture was allowed to equilibrate to room temperature. The supernatant was clarified by passage through a 0.22 μm filter. The supernatant was loaded on to a protein-G column using the GradiFrac system. The column was washed with 5-10 column volumes of binding buffer. The monoclonal antibodies were eluted with the elution buffer and 2 ml fractions were collected. An OD₂₈₀ reading of the fractions were obtained and the fractions containing monoclonal antibodies were pooled. The eluted monoclonal antibody fractions were neutralized by adding 1/20 volume of 3M Tris. The sample was dialyzed in 1×PBS at 4° C. (with 3 buffer changes of at least 3 hours per change). The purified monoclonal antibodies were sterile filtered (0.2 uM) and stored at 2-8° C.

After purification of the mBLA3 monoclonal antibody from the hybridoma supernatant, it was re-tested for binding to HFB cells. The cell samples were prepared as described in Example 4 and incubated with the purified antibody at various concentrations. After incubation the cells were washed, resuspended in 0.1 ml diluent and incubated with 1 μg of FITC conjugated F(ab′)₂ fragment of goat anti-mouse IgG for 30 min at 4° C. The cells were washed, resuspended in 0.5 ml FACS diluent and analyzed using a FACScan cell sorter (Becton Dickinson; San Jose, Calif.). A shift to the right on the FACScan histogram indicated that the purified antibody still bound to the HFB cells.

### Example 4

### Identification and Characterization of Antigen to Which mBLA3 Binds

A cell pellet (approximately 25 ul packed cell volume of the pancreatic tumor cell line CFPAC-1 (ATCC #CRL-1918) or SU.86.86 (ATCC #CRL-1837) was lysed by first diluting the cells to 0.5 ml in water followed by freezing and thawing three times. The solution was centrifuged at 14,000 rpm. The resulting pellet, containing the cell membrane fragments, was resuspended in 50 ul of SDS sample buffer (Invitrogen, Carlsbad, Calif.). The sample was heated at 80° C. for 5 minutes and then centrifuged for 2 minutes at 14,000 rpm to remove any insoluble materials. Other cell lines that express antigen for mBLA3 and can be used for purification include Du-145, HT-29, or the other cell lines which bind mBLA3 as indicated in Table 3.

The samples were analyzed by Western blot using a 4 to 20% Tris-Glycine SDS polyacrylamide non-denaturing gradient gel (Invitrogen; Carlsbad, Calif.) following the manufacturers' directions. Ten microliters of membrane sample were applied to one lane on the polyacrylamide gel. The pre-stained molecular weight markers SeeBlue Plus2 (Invitrogen; Carlsbad, Calif.) were used to assess molecular weight on the gel. The gel proteins were transferred to a nitrocellulose membrane using a transfer buffer of 14.4 g/l glycine, 3 g/l of Tris Base, 10% methanol, and 0.05% SDS. The membranes were blocked, probed with the antibody mBLA3 (at a concentration of 0.5 ug/ml), and developed using the Invitrogen WesternBreeze Chromogenic Kit-AntiMouse according to the manufacturer's directions. In the pancreatic tumor cell membrane samples, one prominent bands were observed migrating at about 120 kDa (+/−10%). Therefore, the mBLA3 antibody recognizes an antigen on a protein in pancreatic tumor cells that, based upon Western blot analysis using this molecular weight marker system, has molecular weights of approximately 120 kDa.

To further characterize antigen for mBLA3, biotinylation of cell surface protein, immunoprecipitation and Western blotting techniques were used. Breast cancer cells SKBR3 (ATCC #HTB30) were grown to confluency on 175 cm² culture dishes. The confluent monolayer was washed three times with Hank's Balanced Salt Solution (HBSS+, containing no sodium bicarbonate, or phenol red; buffered with 10 mM HEPES, pH 7.4, obtained from Sigma Chemicals) and biotinylated with 200 μg of sulfo-NHS-LC-biotin (Pierce Endogen) for 30 minutes at room temperature. The cells were further washed three times with HBSS+containing 0.1M Tris, pH 7.4 (Sigma Chemicals) and incubated in HBSS+containing 0.1M Tris, pH 7.4 for 15 minutes at room temperature. The cells were finally washed three times with HBSS+and lysed by incubating for 5 minutes, on ice, in lysis buffer (HBSS+containing 2% Triton X-100, 2 mM PMSF, 0.1% sodium azide, and 1 tablet per 5 ml lysis buffer of EDTA-free complete mini-protease cocktail (EDTA free complete mini-protease cocktail was obtained from Roche Molecular Biochemicals; all other chemicals were from Sigma Chemicals)). Cells were scraped in lysis buffer and the lysates were collected. Lysates were clarified by centrifugation, 24,000×g for one hour at 4° C. Clarified lysates were first pre-cleared for 2 hours at 4° C. with 5 μl of mouse IgG/BSA conjugated (1 mg/ml) CNBr 4 MB sepharose beads (Amersham Pharmacia). Mouse IgG/BSA beads were removed, and the pre-cleared lysates were then incubated with 5 μl mBLA3 conjugated CNBr 4 MB sepharose beads (conjugated at 1 mg/ml) for 2 hours at 4° C. The mBLA3 beads were removed after the 2 hour incubation. Both the mouse IgG/BSA beads and the mBLA3 beads were individually washed three times with 1 ml of lysis buffer, followed by three 1 ml washes with HBSS+. Washed beads were eluted by the addition of 30 μl of SDS-PAGE sample buffer and boiling at 99° C. for 5 minutes. The samples were resolved on a 4-20% Novex gradient gel (Invitrogen), transferred onto 0.2 μm nitrocellulose (Invitrogen) and visualized by horse radish peroxidase (HRP) conjugated streptavidin (Pierce Endogen) or western blotted with 5 μg/blot of mBLA3. To detect biotinylated cell surface protein that was isolated by mBLA3, the nitrocellulose was stained with HRP conjugated streptavidin. The nitrocellulose was first blocked for 1 hour with blocking buffer (5% non-fat dry milk in phosphate buffered saline containing 0.05% Tween-20 (PBST), Sigma Chemicals). HRP conjugated streptavidin was diluted into PBST at 1 μg/ml and exposed to the nitrocellulose for 30 minutes at room temperature. The nitrocellulose was washed three times with PBST before color development with DAB substrate. For western blotting with mBLA3, the nitrocellulose was similarly blocked for 1 hour in blocking buffer. The nitrocellulose was then incubated in a heat sealed plastic pouch containing 1 ml of 5 μg/ml mBLA3 diluted in blocking buffer. The nitrocellulose was washed 3 times with PBST before incubation with 1 0 ml of 1 μg/ml HRP conjugated donkey anti-mouse IgG (heavy and light chain specific, cross adsorbed against bovine, chicken, goat, guinea pig, syrian hamsters, horse, human, rabbit, sheep serum proteins) for 1 hour at room temp. The nitrocellulose was finally washed three times with PBST and visualized by color development using DAB substrate.

A cell surface protein with molecular weight of 120 kDa was identified using HRP conjugated streptavidin. The protein with the same molecular weight was also recognized in the western blot by mBLA3.

The antigen to which mBLA3 binds was also identified and characterized using the following methods. SKBR3 cells were grown to confluency in 175 cm² flasks. At least 8 175 cm² flasks (total of at least 1400 cm² cell equivalents) of SKBR3 cells were allocated for the purification. 175 cm² of the 1400 cm², or about 12.5% of total cell equivalents were biotinylated in flask with 400 mg/175 cm² of Sulfo-NHS-LC-biotin (Pierce Endogen) in HBSS+(Hank's Balanced Salt Solution containing calcium, magnesium, and 10 mM HEPES (pH 7.2)) for 30 minutes at room temperature. The reaction was quenched by the addition of 1M Tris, pH 8.0 to a final concentration of 100 mM. All SKBR3 cells were then washed with HBSS+before in flask lysis with 1 ml of lysis buffer (HBSS+containing 2% Triton X-100, 1 tablet per 5 ml of Complete-Mini (EDTA free) Protease Inhibitor Cocktail, 2 mM PMSF (Triton X-100, Complete-Mini and PMSF were obtained from Roche Molecular Biochemicals, and 0.03% NaN₃) per 175 cm cell equivalents. The lysates were collected and clarified at 13,000×g for 45 minutes at 4° C. The clarified lysate was pre-absorbed (2 hours at 4° C.) using a 1 ml Mouse IgG/BSA column (1 mg of Mouse IgG and 1 mg of BSA was coupled to 1 ml of activated CNBr 6 MB beads from Pharmacia Biotech). The pre-absorbed lysate was then passed over a 1 ml mBLA3 column (1 mg of mBLA3 was conjugated to 1 ml of activated CNBr 6 MB beads from Pharmacia Biotech) at a flow rate of 5 μl per second at room temp. Both columns were washed with 15 column volumes of lysis buffer followed by 15 column volumes of HBSS+at room temp. The washed column was eluted with 5 column volumes of 0.1M glycine, pH 2.8. Each column volume (1 ml) was added independently, eluates were collected in 1 ml fractions and labeled F1, F2, F3, F4, and F5, with a 30 minutes room temperature incubation occurring between F1 and F2. Prior to sample concentration, F1 was pooled with F4 and labeled as F1+4 while F2 was pooled with F3 and labeled F2+3. F1+4 and F2+3 were independently concentrated on a pre-rinsed Centricon-10 concentrator (Millipore) to a final volume of >400 μl. The concentrated volumes were then further concentrated on a pre-rinsed Microcon-10 concentrator Millipore) until the final volume reaches ˜20 μl.

5% of each concentrated eluate was ran out onto a SDS-PAGE gels (4-20% tris-glycine gradient gels from Invitrogen) under reducing conditions (reduced with 20 mM DTT) and transferred onto nitrocellulose. The blot was then blocked in PBS containing 0.05% Tween-20 (TPBS) and 5% non-fat dried milk for 1 hr at room temp. The blot was resolved using HRP conjugated streptavidin in TPBS for 30 minutes at room temp. Biotinylated proteins were then visualized using DAB. mBLA3 F2+3 showed a strong single band at a molecular weight range of ˜130 kDa. This band was unique when compared to Mouse IgG/BSA F1+4 and F2+3 conditions. mBLA3 F1+4 showed a similar but greatly reduced band. This protein was termed Ag-BLA3.

The remainder of the mBLA3 concentrated eluate was ran out onto a SDS-PAGE gel (4-20% tris-glycine gradient gel from Invitrogen) under reducing conditions and resolved with colour staining using the Gel-Code gel staining reagent (Pierce Endogen). In place of the ˜130 kDa unique band observed in the western blot described above, we observed a unique fuzzy “band” at ˜130kDa concealing two tight bands within ˜5 kDa of each other. Because the signal amplification afforded by the enzymatic development used in western blots can sometimes conceal closely migrating doublets, both bands excised, the sample noted to two closely running proteins, and used for antigen identification.

### Example 5

### Isolation of Antigen of mBLA3 for Mass Spectrometry

Purified antibody mBLA3 was concentrated to approximately 1 mg/ml using a Centricon YM30 concentrator (Millipore Cat. No.4208). Approximately 1 mg of mBLA3 was covalently coupled to 0.35 gram of cyanogen bromide-activated Sepharose 4B resin (Amersham Pharmacia Biotech Cat. No. 17-0430-01) according to the manufacturer's instructions. Freshly grown Rav9926 cells (˜2×10⁹ cells) were harvested from spinner flasks. The cells were pelleted centrifugally, then were resuspended in a total of 15 mL deionized water (dH2O) containing 100 μl of Protease Inhibitor Cocktail (Sigma Cat. No. P8340).

The cell suspension was frozen at −80° C., then thawed. This process was repeated for five cycles in order to disrupt the cells. The cell membranes were collected by centrifugation at 14,000 rpm for 15 minutes at 4° C. in an Eppendorf microcentrifuge.

The cell membrane pellet was resuspended in 2 ml of Hank's Balanced Salt Solution (HBSS, GibcoBRL Cat. No. 14175-079) containing 2% Empigen BB detergent (Calbiochem Cat. No. 324690) and 50 μl Sigma Protease Inhibitor Cocktail, pH 7.0. The cell membrane preparation was then placed on a rotator overnight at 4° C.

The cell membrane preparation was diluted with HBSS to a final concentration of 1% Empigen BB. Insoluble cell debris was removed by centrifugation at 14,000 rpm for 15 minutes at 4° C. in an Eppendorf microcentrifuge. The supernatant containing the soluble membrane proteins was collected and stored at −80° C. until used in affinity purification.

The cell membrane extract was thawed, then mixed with the previously prepared mBLA3-affinity gel and rotated for 2 hours at 4° C. After incubation, the affinity gel was washed extensively as follows: 5 times with serum- and additive-free HBSS+1.0% Empigen BB→3 times with serum- and additive-free HBSS+0.5% Empigen BB→3 times with serum- and additive-free HBSS+0.25% Empigen BB→2 times with serum- and additive-free HBSS+0.125% Empigen BB→2 times with serum- and additive-free HBSS alone→1 time with 0.5 M NaCl in dH₂O→1 time with PBS.

Each wash consisted of 5.0 mL, with the exception of the 0.5 M NaCl wash, which was 1.5 ml. The antigen was then eluted from the affinity gel with 1.5 ml of 2% acetic acid in dH2O for 2 minutes. The 0.5 M NaCl wash and the acid-eluted antigen were retained, and the sample volumes of each were reduced to 100 μl using a SpeedVac (Savant Cat. No. ISS110) on medium heat for ˜2.5 hours.

The samples were then precipitated and extracted by the addition of 400 μL methanol and 100 μL chloroform. Samples were vortexed, then 300 μl dH2O was added and mixed gently. The samples were centrifuged at 14,000 rpm for 4 minutes at room temperature in an Eppendorf microcentrifuge. The protein localizes at the interface of the liquid phases, so most of the top layer was discarded. 400 μl methanol was added to the remainder of the samples and mixed gently. The samples were again centrifuged at 14,000 rpm for 4 minutes at room temperature. The supernatant was discarded, and a SpeedVac was used to dry the samples completely.

The dried samples were reconstituted by the addition of 28 μl 1×LDS sample buffer (Invitrogen Cat. No. NP007) in preparation for electrophoresis. The samples were heated to 75° C. for 10 minutes, then were centrifuged in a microcentrifuge and vortexed to mix. 25 μl of each sample was loaded into a single lane on a pre-cast NuPAGE 4-12% gradient gel (Invitrogen Cat. No. NPO322) for subsequent antigen identification. Two microliters were loaded in another lane for Western blotting analysis. Appropriate molecular weight standards were also included on the gel, as were samples of the cell membrane protein extract before and after incubation with the affinity resin. Electrophoresis was performed according to the manufacturer's instructions. The gel was fixed in 50% methanol containing 10% acetic acid for 30 minutes, then was stained using a Colloidal Blue stain (Invitrogen Cat. No. LC6025) according to the manufacturer's instructions. A non-fixed portion of the gel was transferred onto a nitrocellulose sheet (Invitrogen Cat. No. LC2000) for Western blotting, again according to the manufacturer's instructions. The blot was then probed with mBLA3 and developed using a Western Blotting Kit (Invitrogen Cat. No. WB7103) to confirm antigen recognition.

Stained protein bands from the NuPAGE gel were excised using clean scalpel blades and were placed in clean Eppendorf tubes. Excised bands were stored at −20° C. until used for protein identification by mass spectrometry.

### Example 6

### MALDI Mass Spectrometry

Antigen to which mBLA3 binds was isolated as described in Example 4 and 5 and subject to MALDI mass spectroscopy. Eluates of the immunoaffinity column were separated by SDS-PAGE, and the bands were excised and extracted. The gel slice was tryptically digested “in gel” (Gharahdaghi, F., Weinberg, C. R., Meagher, D. A., Imai, B. S., and Mische, S. M. (1999) Electrophoresis 20, 601-605). Extracted peptides were analyzed by matrix-assisted laser desorption/ionization time-of-flight mass spectrometry (MALDI-Tof) on a Kratos, AXIMA CFR. Peptide masses were determined within 100 ppm and a time ion gate with a curved field reflectron was employed for peptide isolation and fragmentation via post-source-decay (PSD). Searches were conducted with the Protein Prospector Programs (Clauser K. R., Baker P. R. and Burlingame A. L., Analytical Chemistry, Vol. 71, 14, 2871- (1999)) MSFit and MSTag. Sequences matching two major proteins were returned, these proteins were the integrin subunits αv and β6.

### Example 7

### Identification of the Subunit of Integrin αvβ6 that mBLA3 Binds

To determine whether mBLA3 binds to αv, or β6 subunits, or both, purified mBLA3 antigen was fractionated by electrophoresis in SDS gel and transferred onto nitrocellulose membrane. The membranes were probed by Western blot with mBLA3, goat anti-αv integrin C-terminal peptide (Q-20, Santa Cruz Biotechnologies, Cat. No. sc-6617), mouse anti-integrin β6 clone R6G9 (Chemicon international MAB2075Z), mouse anti-integrin αvβ6 clone 10D5 (Chemicon International MAB2077Z), and mouse anti-integrin β6 clone CSβ36 (Chemicon International MAB2076Z). The results showed mBLA3 recognized a single protein band of about 5-10 kD smaller than αv subunit which was recognized by Q-20. Therefore mBLA3 bound to integrin β6 subunit, but did not bind integrin αv subunit. However monoclonal antibodies clone 10D5, R6G9, and CSβ6 recognized neither αv nor β6 band on Western blot, as was consistent with published results that those antibodies does not work in Western blot. To further confirm that mBLA3 antigen is integrin β6, integrin β6 was immunoprecipitated from SK-BR-3 cell lysates using anti-β6 antibodies clones CSB6 and R6G9 were fractionated by electrophoresis in SDS gel and transferred onto nitrocellulose membrane. The membranes were probed by Western blot with mBLA3. Again, mBLA3 recognized a protein band corresponding to β6. In addition, mBLA3 only recognizes non-reduced form of β6 integrin on Western blot. Reduction of β6 integrin subunit with β-mercaptoethanol or dithiothreitol abolished the binding of mBLA3.

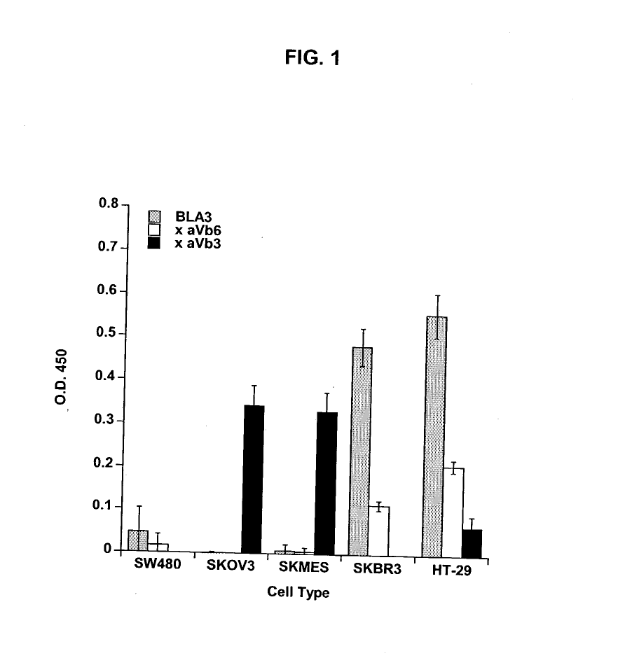

To determine whether mBLA3 cross reacts to integrins other than β6 subunit, SW480 (ATCC #CRL 228), SK-Ov-3 (ATCC #HTB 77), SK-MES-1 (ATCC #HTB 58), SK-BR-3 (ATCC #HTB30), HT-29 (ATCC #HTB 38), and HPAF-II (ATCC #CRL-1997) cell lines were grown to confluence in tissue-culture-treated 96-well plates (Falcon cat no. 354075) in F12/DMEM medium supplemented with 10% fetal bovine serum. Cells were washed with tissue culture medium and then incubated with or without μg/ml mBLA3 in Hank's balanced salt solution (HBSS) containing 1% BSA and 0.1% sodium azide for 1 hour at room temperature. The cells were then washed three times with 100 μl per well of HBSS before incubation with 50 μl per well horse radish peroxidase (HRP) conjugated donkey anti-human IgG heavy and light chain specific antibodies at a concentration of 0.8 μg/ml diluted in HBSS for 30 minutes room temperature. The cells were finally washed three times with HBSS and incubated in 100 μl TMB substrate (KPL Cat No. 50-65-00 and 50-76-01) for 5 minutes and stopped by addition of 100 μl per well of 1μ phosphoric acid. The developed plates were read at O.D. 450 nm. The results were shown in FIG. 1. The binding pattern of mBLA3 in these cell lines was similar to the binding pattern of specific αvβ6 antibodies but distinct from that of specific αvβ3 antibody. There was no specific binding of mBLA3 in SW480 (known cell line expressing α1, α2, α3, α6, αv, and β3 but no β6 and β3, see Koretz K. et al Virchows Arch 425:229-36) nor in SKOV-3 cell line (known cell line expressing α1, α2, α3, α5, α6, αv, β1 and β3 but no β6, see Cannistra SA et al., Gynecol Oncol 58:216-25). There was specific binding of mBLA3 to SK-BR-3, HT-29 and HPAF II cell lines that expressed various levels of integrin β6, consistent with the results obtained with specific αvβ6. Therefore, mBLA3 is an integrin β6 subunit specific monoclonal antibody.

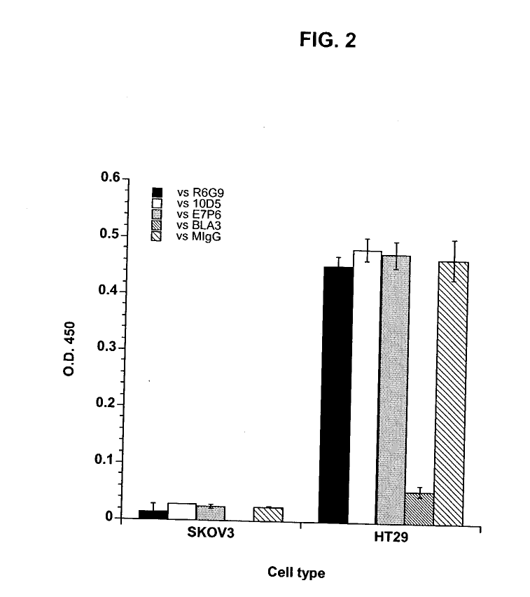

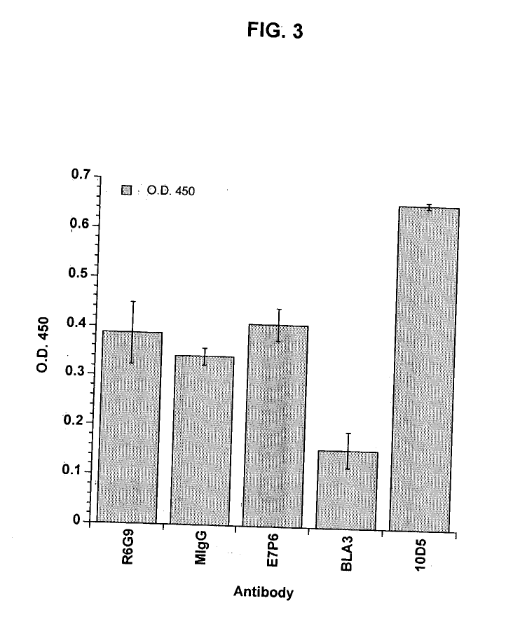

mBLA3 bound to an epitope on β6 integrin subunit that was not recognized by other antibodies commercially available. One of the evidence was that mBLA3 bound to non-reduced SDS denatured β6 subunit but none of the existing monoclonal antibodies did. Another evidence was the competition assay. In this assay, HT-29 cell line was grown to confluence in 96-well tissue culture treated plates in F12/DMEM supplemented with 10% fetal bovine serum. Cells were washed with tissue culture medium and then incubated with or without 0.5 μg/ml biotinylated mBLA3 with or without 10 μg/ml non-labeled mBLA3, R6G9, 10D5, E7P6, or non-specific mouse IgG in Hank's balanced salt solution (HBSS) containing 1% BSA and 0.1% sodium azide for 1 hour at room temperature. The cells were then washed three times with 100 μl per well of HBSS before incubation with 50 μl per well straptavidin-horseradish peroxidase (2ug/ml, Pierce Biochemicals) for 30 minutes room temperature. The cells were finally washed three times with HBSS and incubated in 100 μl TMB substrate (KPL Cat No. 50-65-00 and 50-76-01) for 5 minutes and stopped by addition of 100 μl per well of 1μ phosphoric acid. The developed plates were read at O.D. 450 nm. The results were shown in FIG. 2. No competition was observed between mBLA3 and other available anti-αvβ6 antibodies. A similar assay was also done on mBLA3 purified integrin αvβ6 in a plate ELISA format (FIG. 3). Again, no competition was observed between mBLA3 and the other available anti-αVβ6 antibodies. These data indicate mBLA3 binds to an epitope on β6 subunit of integrin distinct from other available anti-αvβ6 antibodies.

### Example 8

### Inhibition of Cell Adhesion to Fibronectin

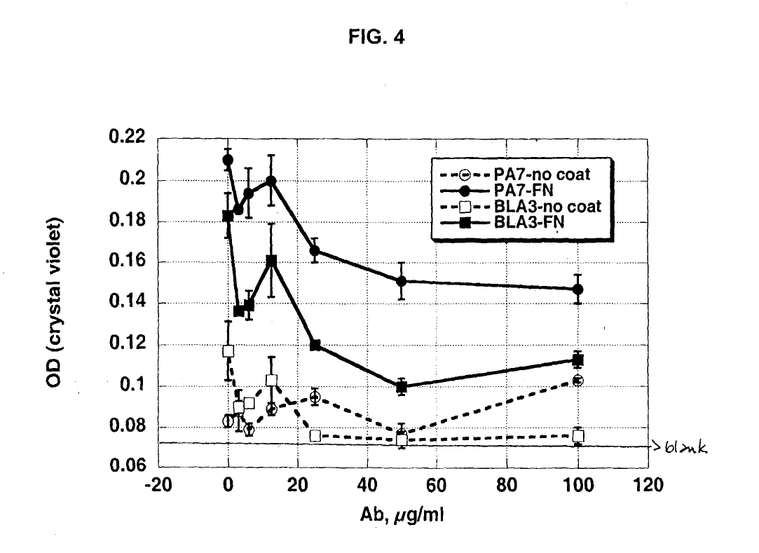

Appropriate wells of a 96 well plate were coated with fibronectin (0.5 μg/well) for 1 hr at room temperature. This was removed and the entire plate was blocked for 30 minutes with 1 mg/ml BSA in DMEM/F12 medium at 37° C. This was removed and antibody was added in DMEM/F12 medium with 1 mg/ml BSA to appropriate wells. SKBR3 cells were removed from the stock tissue culture dish with 10 mM EDTA. The cells were centrifuged and resuspended in DMEM/F12 with 1 mg/ml BSA. The concentration was adjusted such that 1.5×10⁴ cells were added to each well. After 1 hr at 37° C., the dish was blotted on paper towels to remove the medium and unattached cells, washed slowly with 100 μl of PBS/well and blotted again. The cells remaining attached to the dish were stained with crystal violet containing fixative for 10 min. After this was thoroughly washed and the plate was allowed to dry, optical density was read at 540 on a plate reader. As shown in FIG. 4, mBLA3 inhibits SKBR3 adhesion to fibronectin.

### Example 9

### Immunohistochemistry for Binding of mBLA3 Antibody to Tissues with Tumor

Frozen tissue samples were embedded in OCT compound and quick-frozen in isopentane with dry ice. Cryosections were cut with a Leica 3050 CM mictrotome at thickness of 5 μm and thaw-mounted on glass slides. The sections were fixed with ethanol at −20° C. and allowed to air dry overnight at room temperature. The fixed sections were stored at −80° C. until use. For immunohistochemistry, the tissue sections were retrieved and first incubated in blocking buffer (PBS, 5% normal goat serum, 0.1% Tween 20) for 30 minutes at room temperature, and then incubated with the mBLA3 and control monoclonal antibodies diluted in blocking buffer (1 μg/ml) for 120 minutes. The sections were then washed three times with the blocking buffer. The bound monoclonal antibodies were detected with a goat anti-mouse IgG+IgM (H+L) F(ab′)²-peroxidase conjugates and the peroxidase substrate diaminobenzidine (1 mg/ml, Sigma cat. No. D 5637) in 0.1 M sodium acetate buffer pH 5.05 and 0.003% hydrogen peroxide (Sigma cat. No. H1009). The stained slides were counter-stained with hematoxylin and examined under Nikon microscope.

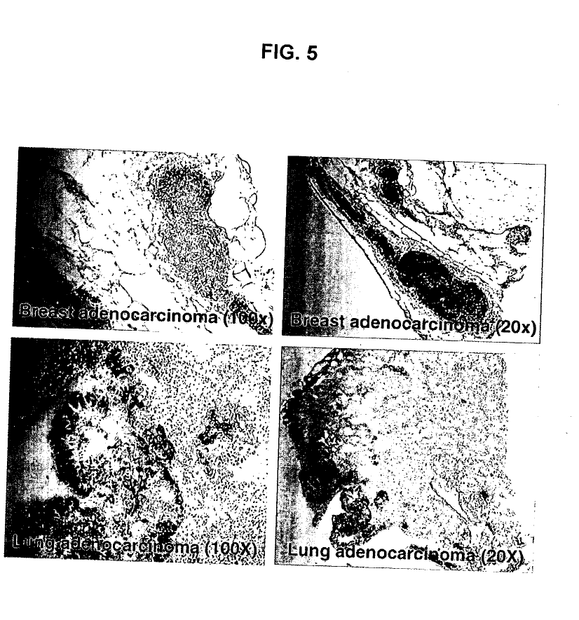

FIG. 5 shows specific binding of mBLA3 antibody (brown color) on a moderately differentiated lung adenocarcinoma and an infiltrating ductal breast adenocarcinoma. Frozen tumor samples were embedded in OCT and sectioned at 6 μm, fixed with ethanol. immunohistochemistry was carried out as described above for frozen sections. Cellular nuclei were counterstained with hematoxylin (pale blue). All cancer cells in the tissues were positive for the cell surface antigen binding mBLA3.

In some cases, paraffin embedded formaldehyde-fixed tissues were used for immunohistochemistry after appropriate antigen retrieval methods were employed. One such antigen retrieval method is described in Mangham and Isaacson, Histopathology 35:129-33 (1999). Other methods of antigen retrieval and/or detection may be used by one skilled in the art. Results from similar experiments performed using frozen tissues or, where appropriate, fixed tissue with antigen retrieval and polyMICA detection were performed. The binding of mBLA3 to a variety of normal and cancer tissues was assessed. In all cases, antibody binding in control fixed tissues was correlated with that of frozen tissues. The results from frozen tissues were only used if the two did not match in the controls. For convenience, Table 1 shows the combined results of the staining of samples of primary tumors representing 5 major types of tumors with mBLA3 using frozen tumor tissues from 5 different sources. For each tumor type, the numbers of tumors testing positive for the antigen of mBLA3, i.e., β6 integrin subunit, and the total number of such tumors tested is shown (+/total). The percentage of tumors binding mBLA3 is also indicated.

TABLE 1
Summary of the incidence of the mBLA3 binding
to integrin αvβ6 on major primary tumor types

| | |
|---|---|
| Colon | 0/8 |
| Lung | 3/9 |
| Breast | 5/8 |
| Prostate | 0/3 |
| Renal | 0/3 |
| Total | 25.8% (8/31) |

Table 2 shows the combined results of the staining of 7 metastatic tumor types with mBLA3 using either fixed or frozen tumor tissues. For each tumor type, the numbers of tumors testing positive for the antigen of mBLA3 and the total number of such tumors tested is shown (+/total). The percentage of tumors binding mBLA3 is also indicated. As shown in Table 2, mBLA3 bound to about 92% of the metastatic tumor types tested. This result, comparing to Table 1, suggested that mBLA3 binding could be used as a marker for metastasis tumor.

TABLE 2
Summary of the incidence of the mBLA3 binding
to integrin αvβ6 on metastatic tumor types

| | |
|---|---|
| Esophageal metastasis | 1/1 |
| Colon metastasis | 0/1 |
| Lung metastasis | 0/1 |
| Breast metastasis | 9/9 |
| Prostate metastasis | 1/1 |
| Renal metastasis | 9/9 |
| Thyroid metastasis | 2/2 |
| Total | 91.7% (22/24) |

### Example 10

### Immunocytochemistry Result from CellArray™

Monoclonal antibody mBLA3 was used to test reactivity with various cell lines from different types of tissues. Cells from different established cell lines were removed from the growth surface without using proteases, packed and embedded in OCT compound. The cells were frozen and sectioned, then stained using a standard IHC protocol. The CellArray™ technology is described in WO 01/43869. Results from the CellArray binding experiments are summarized in Table 3.

TABLE 3
Binding of the mBLA3 antibody to
established human tumor and normal cell lines

| | | | Reactivity |
|---|---|---|---|
| Cell Lines | Organ | Type | with mBLA3 |
| SK-Ov3, END | ovary | cancerous | −, − |
| PC3, LnCAP, Du-145 | prostate | cancerous | −, −, + |
| HT29, SW480 | colon | cancerous | +, − |
| A549, SKMES-1, Rav | lung | cancerous | −, −, −, |
| CA130 | | | |
| PANC-1, Capan-1, | pancreas | cancerous | −, +, +, +, |
| HF700T, CFPAC-1 | | | +, +/−, −, + |
| HPAF-II, AsPC-1, | | | |
| Rav9926, SU.86.86 | | | |
| SKBR3, ZR751, | breast | cancerous | +, +, +, + |
| MCF7, AU565 | | | |
| fhKidney | Renal | Normal fetal | + |
| | | human | |
| HMEC | Human adult | Normal human | + |
| | endothelial cells | adult | |
| NHEK | Normal epidermal | Normal human | − |
| | keratinocyte | | |
| COS-7 | monkey kidney | virus | − |
| | | transformed | |
| AoSMC | aortic smooth | normal | − |
| | muscle | | |
| WI-38 | human fetal lung | normal | − |
| | fibroblasts | | |
| AoSMC | muscle | Aortic smooth | − |
| | | muscle cells | |
| hHEP | Liver | Normal human | − |
| | | heptatocytes | |
| SK-MES-1 | Lung | Human | +/− |
| | | Squamous | |
| | | carcinoma, | |
| | | pleural effusion | |
| f. Bladder | Bladder | Human fetal | + |
| | | bladder | |
| | | epithelial cell | |
| hPED | Pancreas | Human primary | − |
| | | pancreas cell | |
| TR-1 | Testis (Rodent) | capillary | − |
| | | endothelium | |
| | | isolated from | |
| | | testis | |
| Clone 9 | liver Rattus | normal; liver | − |
| | norvegicus (rat) | | |
| CA130 | Lung | Human lung | − |
| | | small cell | |
| | | carcinoma | |
| Hep G2 | Liver | Human | − |
| | | hepatocellular | |
| | | carcinoma | |

### Example 11

### Binding of mBLA3 to Normal Tissues

Normal tissue obtained by surgical resection were frozen and mounted as with tumor tissues. Cryosections were cut with a Leica 3050 CM mictrotome at thickness of 5 μm and thaw-mounted on vectabound-coated slides. The sections were fixed with ethanol at −20° C. and allowed to air dry overnight at room temperature. Primary antibody mBLA3 was used at a dilution of 1 to 100 (final concentration of 1 ug/ml). The tissue sections were retrieved and first incubated in blocking buffer (PBS, 5% normal goat serum, 0.1% Tween 20) for 30 minutes at room temperature, and then incubated with the mBLA3 and control monoclonal antibodies diluted in blocking buffer (1 μg/ml) for 120 minutes. The sections were then washed three times with the blocking buffer. The bound monoclonal antibodies were detected with a goat anti-mouse IgG+IgM (H+L) F(ab′)²-peroxidase conjugates and the peroxidase substrate diaminobenzidine (1 mg/ml, Sigma cat. No. D 5637) in 0.1 M sodium acetate buffer pH 5.05 and 0.003% hydrogen peroxide (Sigma cat. No. H1009). The stained slides were counter-stained with hematoxylin and examined under Nikon microscopePolyMICA™ Detection kit was used to determine binding of mBLA3 to normal skin. The results of staining of normal tissues with mBLA3 is shown in Table 4. Table 5 shows a summary of immunohistochemical staining intensity of individual tumors and normal tissues positive for mBLA3 antigen.

TABLE 4
Distribution of mBLA3 antigen in normal human tissues

| Tissue Type | Results |
|---|---|
| Adrenal | Negative except for 1+ stroma adrenal cortex |
| Breast | Negative except for 1-2+ on glandular epithelium & |
| | myometrium |
| C. cortex | Negative except for 1+ variable vascular endothelium, |
| | 1+ rare glial cells |
| Colon | Negative |
| Duodenum | Negative except for 2+ on Brunner's gland |
| Fallopian Tube | Negative |
| Heart | Negative except for rare 1+ vascular endothelium |
| kidney | Negative except for 2+ variable renal tubules |
| Liver | Negative except for 1+ vascular endothelium |
| Lung | Negative except for 1-2+ variable alveoli, 1+ |
| | endothelium, and 1-2+ variable on bronchial |
| | epithelium |
| Ovary | Negative except for 1+ vascular endothelium |
| Pancreas | Negative except for 1-2+ on pancreatic ducts |
| Spleen | Negative |
| Skeletal Muscle | Negative |
| Skin | Negative except for 2+ eccrine glands, 1+ vascular |
| | endothelium, 2+ hair follicle |
| Stomach | Negative except for 1-2+ gastric epithelium & glands |
| Uterus | Negative except for 1+ vascular endothelium & 1+ |
| | endometrial glands |

The results were scored as ‘+’ for weak positive staining, ‘2+’ for moderate positive staining, ‘3+’ for strong positive staining and ‘−’ for negative staining.

TABLE 5

| | Primary | Metastatic | |
|---|---|---|---|
| Tissue type | carcinoma | carcinoma | Normal tissue |
| prostate | 3 tumors: | 1 tumor: | |
| | −− | +++ | |
| | −− | | |
| | −− | | |
| breast | 4 tumors: | 8 tumors: | Negative except |
| | +/+++ | +++ | for 1-2+ on |
| | + | ++ | glandular |
| | +/++ | +++ | epithelium & |
| | +/+++ | ++ | myometrium |
| | | + | |
| | | +++ | |
| | | +/− | |
| | | +++ | |
| kidney | 3 tumors: | 9 tumors: | Negative except |
| | −− | ++ | for 2+ variable |
| | −− | + | renal tubules |
| | −− | + | |
| | | + | |
| | | + | |
| | | +/− | |
| | | + | |
| | | +/− | |
| | | ++ | |
| thyroid | NA | 2 tumors: | NA |
| | | ++ | |
| | | +/− | |
| Esophagus | 1 tumor: | 1 tumor: | NA |
| | −− | ++ | |

NA: not available

### Example 12

### Effect of mBLA3 on Cell Growth of HT-29 Colorectal Carcinoma Cells and HPAF-II Pancreatic Adenocarcinoma Cells

The ability of the antibodies to reduce cell number in vitro when grown as a monolayer can be assessed using cell monolayers grown in the presence or absence of varying amounts of test or control purified antibody and the change in cell number assessed using MTT. MTT is a dye that measures the activity of mitochondrial enzymes and correlates with relative viable cell number. Cells of interest were plated and grown in F12/DMEM (1:1) growth medium supplemented with 10% fetal bovine serum in 96 well plates. The following cell lines were plated at the following densities in triplicate wells of a 96 well dish: HT-29 and HPAF-II, 10,000 cells/well. Immediately after plating, mBLA3 was added to the medium with different final concentrations ranging from 0 to 50 μg/ml. The cells were incubated at 37° C. in a humidified incubator at 5% CO₂/air for 4 days. At the end of the assay, MTT was dissolved in PBS (5 mg/ml) and added directly to wells at 1:10 dilution. Plates were placed back in incubator for 4 hours. After the incubation, medium was removed and 100 μl DMSO was added to solubilize the MTT precipitate. Plates were read at 540 on plate reader.

An initial experiment indicated that mBLA3 inhibited the growth of the HT-29 colorectal carcinoma cells in a dose dependent manner. However, additional experiments failed to repeat this result. mBLA3 also did not inhibit the growth of HPAF-II pancreatic adenocarcinoma cells at the final concentration of 50 μg/ml.

### Example 13

### Antibody Activity Against Human Tumor Xenografts In Vivo

The inhibitory effect of mBLA3 on human tumor was tested by injecting the antibody into mice with human tumor xenographs. Specifically, HT29 cells (human colon carcinoma line) were grown in F12/DMEM medium supplemented with 10% fetal bovine serum. The cells were removed from the plate, pelleted and 100,000 cells resuspended in 50 μl of neutralized type 1 rat tail collagen. The cell/collagen mixture was allowed to set at 37° C. for 15 minutes and then covered with growth medium and cultured overnight (see Wang, et al. 200 Cancer Research 60:6008-6017). The cells were then grafted beneath the renal capsule of adult male athymic (BALB-nu/nu) mice, with the HT29 on one kidney. The cell grafts were allowed to grow for 7 days. On day 7, 10, and 13, the animals were injected intraperitoneally with 50 mg/kg body weight of mBLA3. The control animal was injected with saline. On day 20 (day 13 after the first injection), the animals were sacrificed, and the cell grafts removed were fixed in paraformaldehyde and embedded in paraffin for sectioning as previously described.

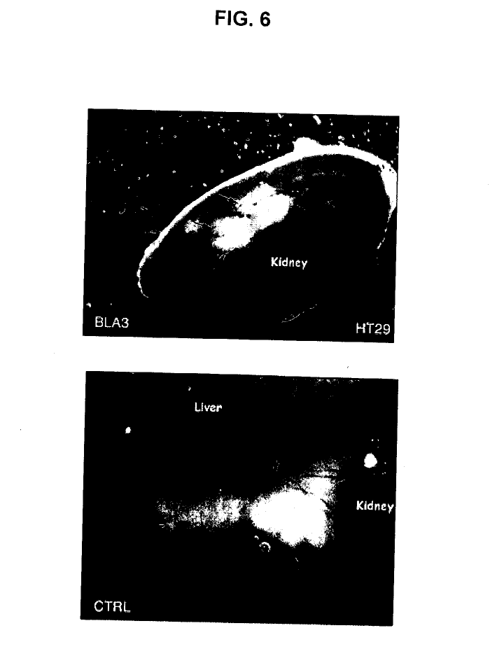

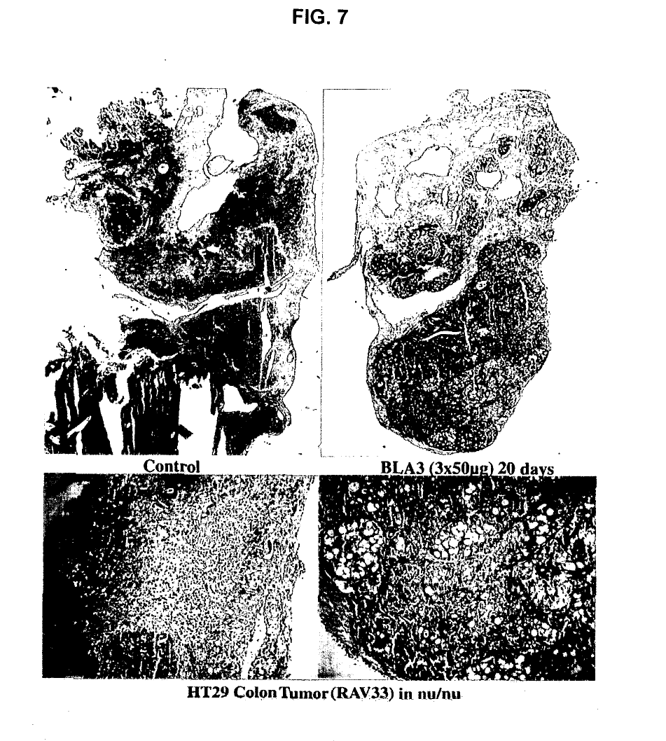

The untreated control showed a large irregular yellowish tumor mass invading the liver (FIG. 6). The mBLA3 treated graft showed a much smaller compact tumor mass with some apoptotic and necrotic areas, and the cells in the graft showed much more differentiated morphology than the control untreated animal (FIGS. 6 and 7). The entire tumor could easily be separated from the adjacent liver in the treated animals, but could not be separated in the untreated controls. In fact, the tumor in the untreated control could be seen to have invaded the liver in the cross section (FIG. 7). These results showed that the mBLA3 antibody was active in causing a decreased tumor mass with much better differentiation and less invasive phenotype, compared to the undifferentiated invasive tumor in the untreated control animal. These results suggested that mBLA3 binding to integrin αvβ6 was sufficient to inhibit and/or kill tumor cells.

### Example 14

### Effect of mBLA3 and Toxin-Conjugated Anti-Mouse IgG on Cancer Cells

Mab-ZAP (Advanced Targeting Systems, San Diego, Calif.) is anti-mouse IgG conjugated to saporin, a toxin which inhibits protein synthesis. This toxin is impermeable to the cell membrane. If a monoclonal antibody is bound to a cell-surface antigen which is internalizable, the toxin-conjugate can bind to the bound monoclonal and be internalized, eventually killing the cell. Being dependent upon internalization for demonstration of toxic activity, the Mab-ZAP can serve to evaluate whether or not a given surface antigen will serve as a suitable target for any toxin that is dependent upon internalization to express cell toxic effects. As such, the Mab-ZAP serves as a model for such internalization-dependent toxins such as maytansinoids, auristatin and calicheamicins.

For testing the internalization of mBLA3 and saporin conjugated anti-mouse IgG by tumor cells and effect of killing the tumor cells after internalization of saporin, human prostate cancer cells, LNCaP (ATCC #CRL-1740) and 22RV1 (ATCC #CRL-2505); human colon cancer cells, HT29 (ATCC #HTB-38); human breast cancer cells, SK-BR-3 (ATCC #HTB30) were removed from stock flasks with 10 mM EDTA and centrifuged. Cells were resuspended at 50,000/ml in appropriate medium and 100 μl plated per well in 96 well plates. Antibody mBLA3 was added immediately to appropriate wells as a 10× concentrate, to make a final concentration of 10 ug/ml. After 15 minutes at room temperature Mab-ZAP (Cat. # IT-04, Advanced Targeting Systems, San Diego Calif.) was added to appropriate wells as 10× concentrate, to make final concentrations from 0.001 pM to 10⁴ pM. After 4 days growth, MTT was added (stock 5 mg/ml PBS, 1:10 dilution in well) for 4 hrs at 37° C. The medium was then removed from all wells and 100 μl/well DMSO was added. The plates was gently swirled to solubilize the blue MTT precipitate and the plates were read in a plate reader at 540 nm.

There was no decrease in MTT staining in human colon cancer cells HT29 and human breast cancer cells SK-BR-3, in the presence of mBLA3 as compared to the staining in the absence of mBLA3 when Mab-ZAP was added from 0.001 to 10⁴ pM, indicating that mBLA3 and Mab-ZAP did not inhibit the growth of these cancer cells under these conditions.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

We claim:

1. An antibody that competitively inhibits preferential binding of an antibody mBLA3 produced by a host cell with a deposit number of ATCC No. PTA-3775 or progeny thereof to β6 integrin subunit.

2. An antibody mBLA3 produced by a host cell with a deposit number of ATCC No. PTA-3775 or progeny thereof.

3. A chimeric antibody comprising variable regions derived from variable regions of a light chain and a heavy chain of the antibody mBLA3 of claim 2 and constant regions derived from constant regions of a light chain and a heavy chain of a human antibody.

4. A humanized antibody of the antibody mBLA3 of claim 2.

5. An antibody according to claims 1, 3, or 4, wherein the antibody is linked to a therapeutic agent.

6. An antibody according to claims 1, 3, or 4, wherein the antibody is linked to a prodrug-activating enzyme which converts a prodrug to an active anti-cancer drug.

7. A host cell line (ATCC No. PTA-3775) or progeny thereof.

8. A complex of β6 integrin subunit bound by an antibody mBLA3 produced by a host cell with a deposit number of ATCC No. PTA-3775 or progeny thereof, or an antibody that competitively inhibits preferential binding of the antibody mBLA3 to β6 integrin subunit.

9. The complex of claim 8, wherein the β6 integrin subunit is on a cancerous cell.

10. The complex of claim 9, wherein the cancerous cell is breast.

11. The complex of claim 9, wherein the cancerous cell is colon.

12. The complex of claim 9, wherein the cancerous cell is lung.

13. The complex of claim 9, wherein the cancerous cell is thyroid.

14. The complex of claim 9, wherein the cancerous cell is pancreatic.

15. The complex of claim 9, wherein the cancerous cell is prostate.

16. The complex of claim 9, wherein the cancer cell is renal.

17. The complex of claim 9, wherein the cancer cell is esophageal.

18. The complex of claim 9, wherein the antibody mBLA3 or the antibody that competitively inhibits preferential binding of the antibody mBLA3 to β6 integrin subunit is linked to a therapeutic agent.

19. The complex of claim 9, wherein the antibody mBLA3 or the antibody that competitively inhibits preferential binding of the antibody mBLA3 to β6 integrin subunit is linked to a prodrug-activating enzyme which converts a prodrug to an active anti-cancer drug.

20. A pharmaceutical composition comprising the antibody of claims 1, 3, or 4, and a pharmaceutically acceptable excipient.

21. A pharmaceutical composition comprising the antibody of claim 5 and a pharmaceutically acceptable excipient.

22. A pharmaceutical composition comprising the antibody of claim 6 and a pharmaceutically acceptable excipient.

23. A kit for detecting cancerous cells comprising an antibody according to any one of claims 1-4.

24. A kit for inhibiting growth of cancerous cells comprising an antibody according to any one of claims 1, 3 and 4.

25. The kit of claim 24, wherein the antibody is linked to a therapeutic agent.

26. The kit of claim 24, wherein the antibody is linked to a prodrug-activating enzyme which converts a prodrug to an active anti-cancer drug.

27. A method of producing the antibody of claim 2 comprising expressing one or more polynucleotides encoding the antibody of claim 2, and purifying the antibody.

28. A method of detecting presence or absence of cancerous cells in an individual comprising contacting cells from the individual with the antibody of claim 1, 2, 3, or 4, and detecting a complex of β6 integrin subunit from the cancerous cells and the antibody, if any.

29. The method of claim 28, wherein the cancerous cells that are detected are breast.

30. The method of claim 28, wherein the cancerous cells that are detected are colon.

31. The method of claim 28, wherein the cancerous cells that are detected are lung.

32. The method of claim 28, wherein the cancerous cells that are detected are pancreatic.

33. The method of claim 28, wherein the cancerous cells that are detected are prostate.

34. The method of claim 28, wherein the cancerous cells that are detected are esophageal.

35. The method of claim 28, wherein the cancerous cells that are detected are thyroid.

36. The method of claim 28, wherein the cancerous cells that are detected are renal.

37. The method of claim 28, wherein the cancerous cells are metastatic.

38. A method of inhibiting growth of cancerous cells in an individual comprising administering to the individual an effective amount of a composition according to claim 20.

39. The method of claim 38, wherein the cancerous cells are colon.

40. A method of delivering a therapeutic agent to cancerous cells in an individual comprising administering to the individual an effective amount of a composition according to claim 21.

41. The method of claim 40, wherein the cancerous cells are colon.

42. A method of delivering a prodrug-activating enzyme to cancerous cells in an individual comprising administering to the individual an effective amount of a composition according to claim 22.

43. The method of claim 42, wherein the cancerous cells are colon.
